(19) United States
(12) Patent Application Publication
(10) Pub. No.: US 2025/0263444 A1
(43) Pub. Date: Aug. 21, 2025

(54) vIRF3-Derived Peptide and Use Thereof for Preventing or Treating Cancer

(71) Applicant: Korea University Research and Business Foundation, Sejong Campus, Sejong (KR)

(72) Inventors: Hye Ra Lee, Sejong (KR); Yeong Jun Kim, Sejong (KR)

(21) Appl. No.: 18/857,042

(22) PCT Filed: May 4, 2023
(86) PCT No.: PCT/KR2023/006165

§ 371 Date: Oct. 15, 2024

(30) Foreign Application Priority Data
May 4, 2022 (KR) 10-2022-0055584

(51) Int. Cl. C07K 14/005; A61K 38/00; A61P 35/00; A61P 35/04; C12Q 1/48

(52) U.S. Cl. CPC C07K 14/005; A61P 35/00; A61P 35/04; C12Q 1/485; A61K 38/00; C12N 2710/16422; C12N 2710/16433; G01N 2333/91215; G01N 2500/20

(57) ABSTRACT

The present invention relates to a peptide derived from Kaposi's sarcoma-associated herpesvirus (KSHV) vIRF3 and a use thereof. According to the present invention, the KSHV vIRF3-derived peptide inhibits the activity of PKM2, reduces the size and volume of tumors, and significantly suppresses the migration and invasion of cancer cells. Therefore, it can be advantageously utilized for the prevention, alleviation, or treatment of cancer or for inhibiting metastasis of cancer cells.

### TECHNICAL FIELD

The present invention relates to a vIRF3-derived peptide and a use thereof for preventing or treating cancer.

### BACKGROUND ART

Chemotherapy is any systemic treatment method using anticancer drugs, and is one of three major cancer treatment methods along with surgery and radiation therapy. Chemotherapy drugs introduced into the body circulate throughout the whole body through the blood vessels and serve to prevent the growth of cancer cells or kill the cancer cells. Traditional anticancer drugs are cytotoxic anticancer drugs that attack rapidly dividing and proliferating cells to block the division of the cells, thereby inducing cancer cell death.

However, the treatment period and number of chemotherapies vary depending on a type of cancer, a type of anticancer drug, response to treatment, and severity of side effects. Moreover, since anticancer drugs are generally cytotoxic drugs, the anticancer drugs act on DNA and microtubules that are normally present in cells, the anticancer drugs show a certain therapeutic effect on cancer cells. However, because the anticancer drugs also have a negative effect on normal cells, the side effects are usually so severe that a rest period of average 2 to 3 weeks is required to wait for normal cells to be restored.

Accordingly, there is a need for the development of anticancer drugs that exhibit effective anticancer effects while having fewer side effects. In this regard, peptide drugs are drugs consisting of 40 or fewer amino acids linked by amide bonds, and are a field expected to have high growth within the biopharmaceutical market due to advantages of not only high biological activity, specificity, and safety, but also ease of synthesis and high productivity.

Meanwhile, pyruvate kinase (PK) is a metabolic enzyme that converts phosphoenolpyruvate into pyruvate during glycolysis. Four PK isoforms exist in mammals: L and R isoforms are expressed in liver and red blood cells, a M1 isoform (PKM1) is expressed in almost all adult tissues, and M2 isoforms (PKM2) are various splice forms of M1 that are expressed during embryonic development. All tumor cells express only an embryonic M2 isoform. A well-known difference between PK isoforms M1 and M2 is that PKM2 is a low-activity enzyme that requires allosteric activation by an upstream glycolytic intermediate such as fructose-1,6-bisphosphate (FBP), whereas PKM1 is a constitutively active enzyme. All tumor cells express only PKM2 of pyruvate kinase that has been proposed as a potential target for cancer therapy. However, PKM2 inhibitors developed to date are not selective, making it difficult to treat diseases related to PKM2 functions.

### DISCLOSURE

### Technical Problem

The present inventors confirmed binding of Kaposi's sarcoma-associated herpesvirus (KSHV) vIRF3 to PKM2, and completed the present invention by confirming that a novel peptide isolated therefrom inhibited PKM2 activity, thereby inhibiting tumor growth and inhibiting migration and invasion.

Therefore, an object of the present invention is to provide a peptide derived from viral interferon regulatory factor 3 (vIRF3) or a fragment thereof.

Another object of the present invention is to provide a pharmaceutical composition for preventing or treating cancer.

Yet another object of the present invention is to provide a method for preventing or treating cancer in a subject other than humans.

Yet another object of the present invention is to provide a pharmaceutical composition for inhibiting migration or metastasis of cancer.

Yet another object of the present invention is to provide a method for screening a cancer therapeutic agent.

Yet another object of the present invention is to provide a quasi-drug for preventing or alleviating cancer.

Yet another object of the present invention is to provide a food composition for preventing or alleviating cancer.

Yet another object of the present invention is to provide a health functional food composition for preventing or alleviating cancer.

Yet another object of the present invention is to provide a method for treating cancer.

Yet another object of the present invention is to provide a method for inhibiting metastasis of cancer.

### Technical Solution

An aspect of the present invention provides a peptide derived from viral interferon regulatory factor 3 (vIRF3) or a fragment thereof, consisting of an amino acid sequence represented by SEQ ID NO: 1.

Another aspect of the present invention provides a pharmaceutical composition for preventing or treating cancer including a vIRF3-derived peptide according to the present invention, a fragment thereof, or a polynucleotide encoding the same.

Yet another aspect of the present invention provides a method for preventing or treating cancer in a subject other than humans, including administering to a subject other than humans a pharmaceutical composition according to the present invention.

Yet another aspect of the present invention provides a pharmaceutical composition for inhibiting migration or metastasis of cancer including a vIRF3-derived peptide according to the present invention, a fragment thereof, or a polynucleotide encoding the same.

Yet another aspect of the present invention provides a method for screening a cancer therapeutic agent, including: (a) contacting a candidate substance with a sample containing PMK2 consisting of an amino acid sequence represented by SEQ ID NO: 7; (b) comparing a physical interaction of the candidate substance with the PKM2 domain with a physical interaction of the vIRF3-derived peptide or the fragment thereof according to claim 1; and (c) selecting the candidate substance as a cancer therapeutic agent if the physical interaction of the candidate substance is similar to or greater than the physical interaction of the vIRF3-derived peptide or the fragment thereof.

Yet another aspect of the present invention provides a quasi-drug for preventing or alleviating cancer including a vIRF3-derived peptide according to the present invention, a fragment thereof, or a polynucleotide encoding the same.

Yet another aspect of the present invention provides a food composition for preventing or alleviating cancer including a vIRF3-derived peptide according to the present invention, a fragment thereof, or a polynucleotide encoding the same.

Yet another aspect of the present invention provides a health functional food composition for preventing or alleviating cancer including a vIRF3-derived peptide according to the present invention, a fragment thereof, or a polynucleotide encoding the same.

Yet another aspect of the present invention provides a method for treating cancer, including treating a subject with a peptide derived from viral interferon regulatory factor 3 (vIRF3) consisting of an amino acid sequence represented by SEQ ID NO: 1, a fragment thereof, or a polynucleotide encoding the same.

Yet another aspect of the present invention provides a method for inhibiting metastasis of cancer, including treating a subject with a peptide derived from viral interferon regulatory factor 3 (vIRF3) consisting of an amino acid sequence represented by SEQ ID NO: 1, a fragment thereof, or a polynucleotide encoding the same.

### Advantageous Effects

The present invention relates to a peptide derived from Kaposi's sarcoma-associated herpesvirus (KSHV) vIRF3 and a use thereof. According to the present invention, the KSHV vIRF3-derived peptide has the effects of inhibiting the activity of PKM2, reducing the size and volume of tumors, and inhibiting the migration and invasion of cancer cells, and thus can be advantageously utilized for the prevention, alleviation or treatment of cancer, or for inhibiting the metastasis of cancer cells.

### DESCRIPTION OF DRAWINGS

FIG. 1A illustrates results of confirming interaction between KSHV vIRF3 and endogenous PKM2, in which an underlined sequence represents PKM2 confirmed by mass-spectrometry analysis of lymphatic endothelial cells (LECs) expressing vIRF3.

FIG. 1B illustrates results of confirming interaction between KSHV vIRF3 and endogenous PKM2, and results obtained by transfecting 293T cells with each plasmid combination and analyzing samples by GST-pull down analysis and then immunoblotting with an anti-HA antibody.

FIG. 1C illustrates results of confirming interaction between KSHV vIRF3 and endogenous PKM2, and results obtained by co-infecting 293T cells with PKM2 and vIRF3 mutants and then immunoprecipitating cell lysates thereof with an anti-HA antibody and performing immunoblotting with anti-GST.

FIG. 1D illustrates results of confirming interaction between KSHV vIRF3 and endogenous PKM2, and results obtained by transfecting 293T cells with PKM2 and vIRF3 or fragments thereof TM1, TM2, and TM3 and then performing immunoprecipitation with an anti-HA antibody and performing immunoblotting with an anti-GST antibody.

FIG. 1E illustrates results of confirming interaction between KSHV vIRF3 and endogenous PKM2, and results obtained by immunoprecipitation with an anti-PKM2 antibody and immunoblotting with an anti-V5 antibody for cell lysates infected with an LEC-expressing pCDH vector, vIRF3, and mutants with deleted aa240-250, aa250-260, aa260-270, and aa270-280 derived from vIRF3, respectively.

FIG. 2A illustrates results of confirming an effect of KSHV vIRF3-PKM2 interaction on PK activity, and results of treating LEC cell lysates expressing pCDH, vIRF3, and vIRF3(Δ250-280) mutants with 0.15% glutaraldehyde for cross-linking of multimeric PKM2, and then performing immunoblotting with indicated antibodies. In this case, β-actin was indicated as an internal loading control.

FIG. 2B illustrates results of confirming the effect of KSHV vIRF3-PKM2 interaction on PK activity and results of fractionating LEC-pCDH and vIRF3 and vIRF3 (A250-280) mutants into cytoplasmic and nuclear fractions and performing immunoblotting. The intensity of the PKM2 band was normalized to the intensity of the internal control band (α-tubulin for the cytoplasm and lamin A/C for a nuclear fraction) and expressed as a comparison with an LEC-pCDH sample. In FIG. 2B, ** indicates p<0.005, *** indicates p<0.0005, and **** indicates p<0.0001.

FIG. 2C illustrates results of confirming the effect of KSHV vIRF3-PKM2 interaction on PK activity and results of lysing cells for indicated metabolite analysis in LECs stably expressing pCDH, vIRF3, and vIRF3 (A250-280) mutants, and performing immunoblotting with a V5 antibody. In this case, β-actin was indicated as an internal loading control.

FIG. 3A illustrates results of confirming whether TCA cycle metabolites increase according to vIRF3, and results of harvesting LECs containing pCDH, vIRF3, and the vIRF3 (A250-280) mutants, lysing the LECs to extract metabolites, and then analyzing the lysate by LC/MS. In FIG. 3A, V represents an experimental group containing pCDH, W represents an experimental group containing vIRF3 (wild type), and M represents an experimental group containing a vIRF3 (A250-280) mutant. In FIG. 3A, NS is p>0.05, ** is p<0.005, *** is p<0.0005, and **** is p<0.0001.

FIG. 3B illustrates results of confirming whether TCA cycle metabolites increase according to vIRF3, in which 13C isotope labeling was performed by treating LECs expressing pCDH, vIRF3, and vIRF3(Δ250-280) mutants with 13C⁶-glucose. The result is a result of treating cells 13C⁶-glucose for 24 hours in a conditioned medium without other carbon sources, harvesting the cells for metabolite extraction, and then performing LC/MS analysis to measure the levels of 13C contained in various metabolites. In FIG. 3B, M+(1 to 6) is a labeling of C13 atoms accumulated from 1 to 6.

FIG. 4A illustrates results of confirming that vIRF3-PKM2 increases total cellular acetyl-CoA to enhance acetylation of SMAD2/3, and results of culturing LECs expressing pCDH, vIRF3, and vIRF3(Δ250-280) mutants with an ACLY inhibitor BMS303141 (right result) or excluding the inhibitor (left result), and then performing immunoblotting using anti-acetyl lysine and anti-V5 antibody. In this case, β-actin was indicated as an internal loading control.

FIG. 4B illustrates results of confirming that vIRF3-PKM2 increases total cellular acetyl-CoA to enhance the acetylation of SMAD2/3 and results of analyzing the acetylation level of SMAD by immunoprecipitation using antibodies targeting each SMAD and then immunoblotting for acetyl-lysine. In this case, β-actin was indicated as an internal loading control.

FIG. 5A illustrates results of confirming vIRF3-mediated acetylation of SMAD2 and SMAD3 and the resulting EndMT of LECs according to treatment with vIRF3 or vIRF3(Δ250-280) mutant, and results of staining LECs stably expressing CDH, vIRF3, and vIRF3(Δ250-280) mutants with antibodies targeting the indicated endothelial or mesenchymal-specific marker proteins.

FIG. 5B illustrates results of confirming vIRF3-mediated acetylation of SMAD2 and SMAD3 and resulting EndMT of LECs according to treatment with vIRF3 or vIRF3(Δ250-280) mutant, and results of performing Boyden's chamber cell migration assay. In FIG. 5B, **** is p<0.0001.

FIG. 5C illustrates results of performing 3D spheroid invasion analysis and analyzing the result using a confocal microscope according to treatment with vIRF3 or vIRF3(Δ250-280) mutant.

FIG. 6A illustrates results of confirming an effect of KSHV vIRF3-PKM2 interaction on KS-like tumor progression in a xenograft model and images of mice 30 days after inoculation with three different types of indicated rKSHV-BAC16. In FIG. 6A, WT means a wild type, MT means vIRF3 (Δ250-280), and Rev.; Rev means revertant-vIRF3(Δ250-280).

FIG. 6B illustrates results of confirming an effect of KSHV vIRF3-PKM2 interaction on KS-like tumor progression in a xenograft model and results of showing average tumor volume, body weight, tumor size, and tumor weight of each experimental group. In FIG. 6B, * indicates p<0.05 and *** indicates p<0.0005.

FIG. 7 illustrates results of confirming mutual-binding between KSHV vIRF3-derived peptide fragments (vIRF3(250-260), vIRF3(260-270), and vIRF3(270-280)) and PKM2.

FIG. 8A illustrates results of confirming that the KSHV vIRF3-derived peptide according to the present invention inhibits KS-like tumor progression by inhibiting PKM2 activity, and results of incubating and lysing LECs expressing vIRF3 with a vehicle, a cell permeable peptide (TAT), or a peptide (TAT-VDP) combining the peptide of the present invention and the cell permeable peptide, and then performing immunoblotting with anti-V5 and anti-PKM2 antibodies. In this case, β-actin was indicated as an internal loading control.

FIG. 8B illustrates results of confirming that the KSHV vIRF3-derived peptide according to the present invention inhibits KS-like tumor progression by inhibiting PKM2 activity, and results of treating and lysing LECs expressing pCDH or vIRF3 with a vehicle, TAT, or TAT-VDP, and then performing immunoblotting with a 4-12% gradient SDS-PAGE gel. In this case, β-actin was indicated as an internal loading control.

FIG. 8C illustrates results of confirming that the KSHV vIRF3-derived peptide according to the present invention inhibits KS-like tumor progression by inhibiting PKM2 activity, and results of treating LECs expressing pCDH or vIRF3 with a vehicle, TAT, or TAT-VDP, and then measuring cellular acetyl-CoA and ATP levels thereof. In FIG. 8C, NS is p>0.05, * is p<0.05, and **** is p<0.0001.

FIG. 8D illustrates results of confirming that the KSHV vIRF3-derived peptide according to the present invention inhibits KS-like tumor progression by inhibiting PKM2 activity, and results of inoculating LECs expressing pCDH vector or vIRF3 into a chick embryo chorioallantoic membrane (CAM), treating the LECs with a vehicle, TAT or TAT-VDP, harvesting the CAM samples and performing immunohistochemical analysis. H&E staining for each tissue sample was indicated as control staining. In FIG. 8D, EC represents ectoderm, M represents mesoderm, and ED represents endoderm.

FIG. 8E illustrates results of confirming that the KSHV vIRF3-derived peptide according to the present invention inhibits KS-like tumor progression by inhibiting PKM2 activity, and results of monitoring the average tumor volume and body weight of each experimental group for 30 to 60 days (top, (a) represents an experimental group treated with TAT-VDP 30 days after TAT treatment, (b) represents an experimental group treated with TAT-VDP, and (c) represents a positive control group treated with TAT only.) and results illustrating representative photographs of tumor and tumor weight of each experimental group (bottom). In FIG. 8E, ** is p<0.005 and N.D. means not determined.

### MODES OF THE INVENTION

Hereinafter, the present invention will be described in detail.

The present invention provides a peptide derived from viral interferon regulatory factor 3 (vIRF3) or a fragment thereof, consisting of an amino acid sequence represented by SEQ ID NO: 1.

In the present invention, the vIRF3 may be Kaposi's sarcoma-associated herpesvirus (KSHV) vIRF3, and may be Genebank: MZ923827.1, and may consist of an amino acid sequence represented by SEQ ID NO: 2.

The KSHV is one of seven types of human cancer viruses called oncovirus, and also the eighth type of human herpesvirus. The name HHV-8 is commonly used. The virus not only causes Kaposi's sarcoma (KS), which commonly occurs in patients with acquired immune deficiency syndrome (AIDS), but also causes primary effusion lymphoma (PEL) and multicentric Castleman's disease.

The vIRF3-derived peptide consisting of the amino acid sequence represented by SEQ ID NO: 1 is an amino acid sequence included within aa 250-280 of vIRF3 consisting of an amino acid sequence represented by the SEQ ID NO: 2, and according to an embodiment of the present invention, it was confirmed that vIRF3₂₅₀₋₂₈₀ specifically binds to PKM₂₂₁₀₋₄₀₂. Furthermore, it was confirmed that all mutants vIRF3(250-260), vIRF3(260-270), and vIRF3(270-280) expressing 11 fragment peptides of amino acids at positions 250 to 280 of vIRF3 mutual-bound with PKM2.

Therefore, in the present invention, the fragment may be at least one selected from the group consisting of vIRF3 aa 250-260, vIRF3 aa 260-270, and vIRF3 aa 270-280 within the amino acid sequence of the vIRF3-derived peptide consisting of the amino acid sequence represented by the SEQ ID NO: 1. Preferably, the vIRF3 aa 250-260 may consist of an amino acid sequence represented by SEQ ID NO: 3, the vIRF3 aa 260-270 may consist of an amino acid sequence represented by SEQ ID NO: 4, and the vIRF3 270-280 may consist of an amino acid sequence represented by SEQ ID NO: 5.

A polypeptide including the amino acid sequence of the present invention may be prepared by expressing a polynucleotide encoding the polypeptide sequence of the present invention in a suitable host cell. This may be performed by methods of recombinant DNA technology known to those skilled in the art.

In addition, the peptide according to the present invention may be prepared by chemical synthesis known in the art. Representative methods include liquid or solid phase synthesis, fragment condensation, and 9-fluorenylmethoxycarbonyl (F-MOC) or tert-butyloxycarbonyl (T-BOC) chemistry, but are not necessarily limited thereto. In addition, the peptide of the present invention may be prepared by genetic engineering methods. First, a DNA sequence encoding the peptide is constructed using a conventional method. The DNA sequence may be constructed by PCR amplification using appropriate primers. As another method, the DNA sequence may be synthesized by standard methods known in the art, for example, using an automated DNA synthesizer (e.g., those sold by Biosearch or Applied Biosystems Co., Ltd.). The constructed DNA sequence is inserted into a vector containing one or more expression control sequences (e.g., promoter, enhancer, etc.) that are operatively linked to the DNA sequence to control the expression of the DNA sequence thereof, and a host cell is transformed with a recombinant expression vector formed therefrom. The produced transformant is cultured in appropriate medium and conditions to express the DNA sequence, and a substantially pure peptide encoded by the DNA sequence is recovered from the culture. The recovery may be performed using methods (e.g., chromatography) known in the art. The “substantially pure peptide” means that the peptide according to the present invention does not substantially include any other protein derived from the host. In addition, the peptide according to the present invention may be in a form in which an N-terminus or C-terminus is modified or protected with various organic groups, in order to protect from protein cleavage enzymes in a living body and increase stability. That is, the C-terminus of the peptide is not particularly limited as long as it may be modified to increase the stability, but may be preferably modified with a hydroxyl group (—OH) or an amino group (—NH₂). In addition, the N-terminus of the peptide is not particularly limited as long as it may be modified to increase the stability, but preferably, may be modified with a group selected from the group consisting of an acetyl group, a fluorenyl methoxy carbonyl (Fmoc) group, a formyl group, a palmitoyl group, a myristyl group, a stearyl group, and a polyethylene glycol (PEG).

In addition, the peptide according to the present invention may have any one amino acid among peptide mimetics including D-type, L-type, peptoid monomers or unnatural amino acids.

In addition, the peptide according to the present invention may be a dimer, a trimer or a multimer, and may include various functional groups at the N-terminus or C-terminus.

As examples of the various functional groups, the functional group at the N-terminus may be any one selected from the group consisting of a free amine, acetylation, biotin, and a fluorophore, and the functional group at the C-terminus may be any one selected from the group consisting of a free acid, amidation, biotin, and a fluorophore, but is not limited thereto.

It will be apparent to those skilled in the art that biologically functional equivalents that may be included in the range of the vIRF3-derived peptides of the present invention or fragments thereof will be limited to include mutants in the amino acid sequence that exhibit equivalent biological activity to the peptide of the present invention.

These amino acid mutants are made based on the relative similarity of amino acid side-chain substituents, such as hydrophobicity, hydrophilicity, charges, sizes and the like. By analysis of the size, shape and type of the amino acid side-chain substituent, it can be seen that arginine, lysine and histidine are all positively charged residues; alanine, glycine and serine have similar sizes; and phenylalanine, tryptophan and tyrosine have similar shapes. Thus, based on these considerations, arginine, lysine and histidine; alanine, glycine and serine; and phenylalanine, tryptophan and tyrosine may be biologically functional equivalents.

In introducing mutation, a hydrophobic index of amino acids may be considered. Each amino acid is assigned with a hydrophobic index according to its hydrophobicity and charges: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The hydrophobic amino acid index is very important in assigning an interactive biological function of the peptide. It is known that substitution with amino acids having similar hydrophobic indexes may retain similar biological activity. When introducing mutations with reference to the hydrophobic index, substitution is made between amino acids which exhibit a hydrophobic index difference, preferably within ±2, more preferably within ±1, and much more preferably within ±0.5.

Meanwhile, it is also well known that substitutions between amino acids having similar hydrophilic values result in peptides with equivalent biological activity. As disclosed in US Patent Registration No. 4,554,101, the following hydrophilic values are assigned to each amino acid residue: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

When introducing mutations with reference to the hydrophilic index, substitution is made between amino acids which exhibit a hydrophilic index difference, preferably within ±2, more preferably within ±1, and much more preferably within ±0.5.

Amino acid exchange in peptides without changing the activity of the molecule as a whole is known in the art. The most commonly occurring exchange is an exchange between amino acid residues Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Considering the mutants having the above-described biologically equivalent activity, the vIRF3-derived peptide of the present invention or the fragment thereof is interpreted to include a sequence representing substantial identity with a sequence described in a sequence list. The substantial identity means a sequence showing at least 80% homology, more preferably 90% or more homology, when the peptide sequence of the present invention and any other sequence are aligned to correspond to each other as much as possible and the aligned sequence is analyzed using an algorithm commonly used in the art. Any alignment method known in the art may be used without limitation for sequence comparison.

In addition, the vIRF3-derived peptide according to the present invention may further include a cell permeable domain to enhance cell permeability. The cell permeable domain may refer to a short peptide that promotes cellular uptake of various molecular cargoes (small chemical molecules to nano-sized particles and large fragments of DNA). A “cargo” such as a protein is linked to a peptide either through covalent chemical bonds or through non-covalent interactions. The function of the cell permeable domain is to deliver the cargo into the cell, and a process that typically occurs through endocytosis with a cargo delivered to endosomes in living mammalian cells. The cell permeable domain typically has an amino acid composition containing a high relative abundance of positively charged amino acid, such as lysine or arginine, or has a sequence containing alternate patterns of polar/charged amino acids and nonpolar, hydrophobic amino acids. In the present invention, the cell permeable domain may be an HIV TAT peptide, but is not limited thereto.

For example, a vIRF3-derived peptide additionally including an HIV TAT peptide as the cell permeable domain may consist of an amino acid sequence represented by SEQ ID NO: 6. The vIRF3-derived peptide additionally including the HIV TAT peptide may also be interpreted as including a sequence showing substantial identity with the amino acid sequence represented by SEQ ID NO: 6, for the same reasons as above.

According to another embodiment of the present invention, as a result of treating a LEC cell line expressing vIRF3 with the vIRF3-derived peptide consisting of the amino acid sequence represented by SEQ ID NO: 1, it was confirmed that PKM2 activity was significantly inhibited. In addition, according to yet another embodiment of the present invention, as a result of subcutaneously injecting the vIRF3-derived peptide consisting of an amino acid sequence represented by SEQ ID NO: 1 according to the present invention into an animal model transplanted with LECs infected with KSHV, it was confirmed that the size and volume of tumor were significantly reduced.

Therefore, the present invention provides a pharmaceutical composition for preventing or treating cancer including a vIRF3-derived peptide according to the present invention, a fragment thereof, or a polynucleotide encoding the same.

As used herein, the term “treatment” refers to an approach to obtaining beneficial or desirable clinical results, and includes alleviation of symptoms, reduction in disease degree, a stabilized (i.e., not worsening) state of the disease, delay or reduction in rate of disease progression, alleviation or temporary palliation and reduction of the disease state, regardless of detecting or not a beneficial or desirable clinical results and partially or entirely for the purposes of the present invention, but is not limited thereto. Accordingly, the treatment refers to both curative and preventive treatments, and conditions to be treated include not only conditions that already have a disease, but also conditions to be prevented. In addition, the treatment may mean all actions that improve or benefit the symptoms of cancer by administering the pharmaceutical composition of the present invention to a subject.

As used herein, the term “prevention” refers to all actions that inhibit or delay the onset of a disease for which the composition is effective by administering or applying the composition according to the present invention. It will be apparent to those skilled in the art that the composition of the present invention having a preventive or therapeutic effect on cancer may prevent these diseases when taken or administered at the initial symptoms of cancer or before the appearance of such symptoms.

The pharmaceutical composition of the present invention may be administered to a “subject” that has developed or is likely to develop cancer, and the “subject” may mean all animals including humans.

In addition, according to yet another embodiment of the present invention, as a result of administering the vIRF3-derived peptide according to the present invention to a chicken embryo chorioallantoic membrane (CAM) inoculated with a LEC cell line exhibiting high PKM2 activity, it was confirmed that the migration and invasion of cancer cells were significantly inhibited.

Therefore, the present invention provides a pharmaceutical composition for preventing migration or metastasis of cancer including a vIRF3-derived peptide according to the present invention, a fragment thereof, or a polynucleotide encoding the same.

The pharmaceutical composition of the present invention may be formulated and used in the form of oral formulations such as powders, granules, tablets, capsules, suspensions, emulsions, syrups, aerosols, etc., external preparations, suppositories, and sterile injectable solutions according to conventional methods, respectively. The carrier, the excipient, and the diluent that may be included in the pharmaceutical composition may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. When the pharmaceutical composition is formulated, the formulation may be prepared by using diluents or excipients, such as a filler, an extender, a binder, a wetting agent, a disintegrating agent, and a surfactant, which are generally used. Solid formulations for oral administration include tablets, pills, powders, granules, capsules, and the like, and these solid formulations may be prepared by mixing at least one excipient, for example, starch, calcium carbonate, sucrose or lactose, gelatin, and the like with the vIRF3-derived peptide according to the present invention, the fragment thereof, or the polynucleotide encoding the same. Further, lubricants such as magnesium stearate and talc are used in addition to simple excipients. Liquid formulations for oral administration may correspond to suspensions, oral liquids, emulsions, syrups, and the like, and may include various excipients, for example, a wetting agent, a sweetener, an aromatic agent, a preservative, and the like, in addition to water and liquid paraffin which are commonly used as simple diluents. Formulations for parenteral administration include sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

The dose of the pharmaceutical composition of the present invention will vary depending on the age, sex, and weight of a subject to be treated, a specific disease or pathological condition to be treated, the severity of the disease or pathological condition, a route of administration, and the judgment of a prescriber. The dose based on these factors is determined within a level of those skilled in the art, and in general, the dose is in the range of 0.01 mg/kg/day to about 2000 mg/kg/day. A more preferable dose is 0.1 mg/kg/day to 1000 mg/kg/day. The administration may be performed once a day or several times a day. The dose does not limit the scope of the present invention in any aspect.

The pharmaceutical composition of the present invention may be administered to mammals such as mice, livestock, and humans via various routes, and may be administered both parenterally and orally, but preferably administered parenterally.

Forms for parenteral administration may include toothpaste, mouthwash, and topical administration agents (creams, ointments, dressing solutions, sprays, and other topical agents). As an example of the formulation of the topical administration agent, the pharmaceutical composition for preventing or treating cancer according to the present invention may be formulated to be fixed to a film or patch containing a water-soluble polymer and attached to a region around the onset of cancer.

In the present invention, the pharmaceutical composition for preventing or treating cancer or the pharmaceutical composition for inhibiting the migration or metastasis of cancer may further include any compound or natural extract which has already been verified in safety to increase the prevention, treatment, movement and invasion inhibition effects of cancer and is known to have prevention, treatment, or migration and invasion inhibition effects of cancer, in addition to the vIRF3-derived peptide according to the present invention, the fragment thereof or the polynucleotide encoding the same as an active ingredient.

In addition, the pharmaceutical composition for preventing or treating cancer or the pharmaceutical composition for inhibiting the migration or metastasis of cancer of the present invention may be used together with surgical treatment of cancer.

In the present invention, the cancer may be a cancer that overexpresses PKM2. The cancer overexpressing PKM2 may be cancer infected with KSHV or cancer that is not infected with KSHV. In addition, examples of the cancer overexpressing PKM2 include lung cancer, prostate cancer, colon cancer, breast cancer, lymphoma, kidney cancer, pancreatic cancer, liver cancer, ovarian cancer, and gallbladder cancer, but are not limited thereto.

In addition, the present invention also provides a method for preventing or treating cancer in a subject other than humans, including administering to a subject other than humans a pharmaceutical composition according to the present invention.

The subject refers to a subject suspected of having cancer or having the potential to have cancer, and includes non-human animals such as monkeys, dogs, cats, rabbits, guinea pigs, rats, mice, cows, sheep, pigs, goats, birds, and fish.

Further, the present invention provides a method for screening a cancer therapeutic agent, including: (a) contacting a candidate substance with a sample containing PMK2 consisting of an amino acid sequence represented by SEQ ID NO: 7; (b) comparing a physical interaction of the candidate substance with the PKM2 domain with a physical interaction of the vIRF3-derived peptide or the fragment thereof according to claim 1; and (c) selecting the candidate substance as a cancer therapeutic agent if the physical interaction of the candidate substance is similar to or greater than the physical interaction of the vIRF3-derived peptide or the fragment thereof.

The candidate substance refers to an unknown substance used in screening to examine whether to affect the activity of PMK2 consisting of an amino acid sequence represented by SEQ ID NO: 7. The candidate substance may include chemicals, antisense oligonucleotides, antisense-RNA, siRNA, shRNA, miRNA, specific antibodies, or natural product extracts, but is not limited thereto.

In the present invention, the “sample” is used in the broadest sense. Meanwhile, the sample means including specimens or cultures (e.g., microorganism cultures). On the other hand, the sample means including both biological and environmental samples. In addition, the sample may include synthetic-origin specimens. The biological sample may be at least one selected from the group consisting of whole blood, plasma, serum, sputum, tears, mucus, nasal washes, nasal aspirate, breath, urine, semen, saliva, peritoneal washings, ascites, cystic fluid, meningeal fluid, amniotic fluid, leukocytes, peripheral blood mononuclear cells, buffy coat, glandular fluid, pancreatic fluid, lymph fluid, pleural fluid, nipple aspirate, bronchial aspirate, synovial fluid, joint aspirate, organ secretions, cells, cell extract and cerebrospinal fluid, but is not limited thereto.

In addition, the present invention provides a quasi-drug for preventing or alleviating cancer including a vIRF3-derived peptide according to the present invention, a fragment thereof, or a polynucleotide encoding the same.

In the present invention, the “quasi-drug” refers to products with a milder action than drugs among products used for the purpose of diagnosing, treating, alleviating, mitigating, handling or preventing human or animal diseases. For example, according to the Pharmaceutical Affairs Act, the quasi-drug includes products used for the treatment or prevention of human/animal diseases, products with mild or no direct action on the human body, etc., by excluding products used for the purpose of drugs.

When used as a quasi-drug additive, the vIRF3-derived peptide according to the present invention, the fragment thereof, or the polynucleotide encoding the same may be added as it is or used together with other quasi-drugs or quasi-drug ingredients, and may be appropriately used according to a conventional method. The mixing ratio with other ingredients used together may be appropriately determined depending on the purpose of use (e.g., preventive, alleviation, or therapeutic treatment).

In the present invention, the quasi-drug composition may be formulated as an ointment, a cream, a gel, a lotion, a solution, a dressing, a patch, a blister, a tape, a mist, an external powder, a spray, etc.

In addition, the present invention provides a food composition for preventing or alleviating cancer including a vIRF3-derived peptide according to the present invention, a fragment thereof, or a polynucleotide encoding the same.

In addition, the present invention provides a health functional food composition for preventing or alleviating cancer including a vIRF3-derived peptide according to the present invention, a fragment thereof, or a polynucleotide encoding the same.

The food composition according to the present invention includes all forms, such as functional foods, nutritional supplements, health foods, and food additives.

As used herein, the term “health functional food” refers to food manufactured and processed by using a specific ingredient for health supplementation as a raw material or extracting, concentrating, purifying, mixing, etc., a specific ingredient contained in a food raw material, and refers to food designed and processed to sufficiently exhibit in the body biological control functions such as biological defense, regulation of biological rhythm, prevention and restoration of disease, etc. by the ingredients, and refers to food that may perform functions related to prevention of disease or health restoration.

The food composition or the health functional food composition according to the present invention may be prepared in various forms according to general methods known in the art.

For example, as the health food, the vIRF3-derived peptide according to the present invention, the fragment thereof, or the polynucleotide encoding the same may be granulated, encapsulated and powdered to be ingested or prepared in the form of tea, juice and drink to be drunk. In addition, the vIRF3-derived peptide according to the present invention, the fragment thereof, or the polynucleotide encoding the same may be mixed with known substances or active ingredients known to have the prevention, alleviation, or treatment effect of cancer to be prepared in the form of a composition.

In addition, the functional food may be prepared by adding a periodontal ligament stem cell culture medium cultured in a medium containing metformin of the present invention to beverages (including alcoholic beverages), fruits and processed foods thereof (e.g., canned fruit, bottled food, jam, marmalade, etc.), fish, meat and processed foods thereof (e.g., ham, sausage, corned beef, etc.), bread and noodles (e.g., udon, buckwheat noodles, ramen, spaghetti, macaroni, etc.), fruit juice, various drinks, cookies, sweets, dairy products (e.g., butter, cheese, etc.), edible vegetable oil, margarine, vegetable protein, retort food, frozen food, various seasonings (e.g., soybean paste, soy sauce, sauce, etc.), etc.

The preferred content of the vIRF3-derived peptide consisting of the amino acid sequence represented by SEQ ID NO: 1 according to the present invention, the fragment thereof, or the polynucleotide encoding the same in the food composition of the present invention is not limited thereto, but may be, for example, 0.01 to 80 wt % of the finally manufactured food, and preferably 0.01 to 50 wt % of the finally manufactured food.

In addition, in order to use the food composition of the present invention in the form of food additives, the composition may be prepared and used in the form of powders or concentrates.

Further, the present invention provides a method for treating cancer, including treating a subject with a peptide derived from viral interferon regulatory factor 3 (vIRF3) consisting of an amino acid sequence represented by SEQ ID NO: 1, a fragment thereof, or a polynucleotide encoding the same.

Furthermore, the present invention provides a method for inhibiting metastasis of cancer, including treating a subject with a peptide derived from viral interferon regulatory factor 3 (vIRF3) consisting of an amino acid sequence represented by SEQ ID NO: 1, a fragment thereof, or a polynucleotide encoding the same.

The above-described contents of the present invention are equally applied to each other unless otherwise contradict each other, and those appropriately modified and implemented by those skilled in the art are also included in the scope of the present invention.

Hereinafter, the present invention will be described in detail through Examples, but the scope of the present invention is not limited only to the Examples below.

Experimental Example 1. Purchase of Chemicals and Antibodies (Anti-Acetyl H3)

Doxycycline (DOX), sodium butyrate (NaB), and D-glucose solution were purchased from Sigma. A PKM2 inhibitor compound 3k was purchased from Selleckchem, and carbonyl cyanide 4-(trifluoromethoxy)phenylhydrazone (FCCP), Rotenone, Antimycin A1, 2-deoxy-D-glucose (2-DG), and BMS-303141 were purchased from Cayman Chemical. An Oligomycin complex was purchased from Biovision, and primary antibodies were purchased from the following manufacturers: Anti-acetylated-lysine (6952S) was purchased from Cell signaling technology, and anti-KSHV LANA (LNA-1; 4013) and anti-Guinea pig IgG-Alexa Fluor®568 (ab-175714) were purchased from Abcam. Anti-MCL-1 (sc-12756), anti-GST (sc-138), anti-Lamin A/C (sc-625), anti-ORF45 (sc-53883), anti-K8.1 (sc-65446), anti-SMAD2 (sc-101153), anti-SMAD3 (sc-101154), anti-Vimentin (sc-6260), anti-Fibronectin (sc-8422), anti-N-cadherin (sc-59987), anti-α-SMA (sc-53142), and anti-acetyl H3 (sc-32268) were purchased from Santa Cruz. Anti-β-actin (A-5441), anti-FLAG (F1804), and anti-tubulin (GTU-88) were purchased from Sigma. Anti-V5 (MA5-15253) was purchased from Invitrogen, anti-VE-cadherin (555661) was purchased from BD Pharmingen, and anti-HA (M180-3) was purchased from MBL. Anti-PKM1 (15821-1-AP) and anti-PKM2 (15822-1-AP) were purchased from Proteintech, anti-SMAD7 (NBP2-24710) and anti-LANA2 (NB200-167) were purchased from Novus biologicals, and anti-alpha smooth muscle actin (449004) was procured from Synaptic systems.

### Experimental Example 2. Synthesis of KSHV vIRF3-Derived Peptide and Binding of Cell Permeable Peptide Thereto

A KSHV vRIF3-derived 250-280 peptide, a KSHV vRIF3-derived 250-260 peptide, a KSHV vRIF3-derived 260-270 peptide, and a KSHV vRIF3-derived 270-280 peptide were requested and synthesized by Bionics Co., Ltd.

### Experimental Example 3. Analysis Method

### 3-1. Cell Culture and Cell Line Construction

293T, SLK, and iSLK cells were cultured in a Dulbecco's modified Eagle's medium (DMEM) containing 10% fetal bovine serum (FBS) and 1% P(penicillin)/S(streptomycin) (Corning). LEC-E6/E7 cells were maintained in a VascuLife® VEGF endothelial medium complete kit (Lifeline cell technology) together with 200 μg/ml G418. To establish lymphatic endothelial cells (LECs) expressing viral interferon regulatory factor 3 (vIRF3), E6/E7 immortalized LECs were prepared as follows. First, the viral supernatant obtained from a retrovirus producing cell line PA317-Lxsn expressing HPV16 E6/E7 was inoculated into pre-cultured LECs together with polyethylene, and then treated with a selective marker G418 for 2 weeks to selectively incubate and prepare only LECs infected with retrovirus. Through this, immortalized LECs expressing E6/E7 were obtained. The obtained LECs were infected with a V5/vIRF3 expressing lentivirus using a method known in the art. 48 hours after infection, cells were selected using puromycin (0.5 μg/ml) and G418 (200 μg/ml). Transient transfection was performed using polyethylenimine hydrochloride (PEI) (Sigma) or FuGENE HD (Promega) according to the manufacturers' protocols.

### 3-2. Plasmid Construction

A pCMV-HA-hPKM2 plasmid was provided by from the Miyagi Cancer Center Research Institute, Natori, Japan. In addition, a vIRF3 (Δ250-280) mutant was generated by sequentially deleting 10 amino acids from alanine 250 using a QuikChange XL site-directed mutagenesis kit (Agilent). Primer pairs used in vIRF3 deletion mutation were as follows: vIRF3(Δ250-260) forward 5′-GCTTGATAAAGAATCTATGGACATGCTAATGA-3′ (SEQ ID NO: 8), vIRF3(Δ250-260) reverse 5′-TCATTAGCATGTCCATAGATTCTTTATCAAGC-3′ (SEQ ID NO: 9), vIRF3(Δ260-270) forward 5′-GCTTGATAAAGAATCTGAAGACCTCTTTGATC-3′ (SEQ ID NO: 10), vIRF3(Δ260-270) reverse 5′-GATCAAAGGTCTTCAGATTCTTTATCAAGC-3′ (SEQ ID NO: 11), vIRF3(Δ270-280) forward 5′-GCTTGATAAAGAATCTGAGGATGTTATCGCAA-3′ (SEQ ID NO: 12), and vIRF3(Δ270-280) reverse 5′-TTGCGATAACATCCTCAGATTCTTTATCAAGC-3′ (SEQ ID NO: 13). GST-tagged vIRF3 250-260, 260-270, and 270-280 were subcloned into a pEBG-GST vector with BamHI and NotI restriction sites using the following primers: vIRF3(250-260) forward 5′-GATCCGCCACCATGGCATGTGCATTGATGTACCACGTGGGACAGGAGTA GGC-3′ (SEQ ID NO: 14), vIRF3(250-260) reverse 5′-GGCCGCCTACTCCTGTCCCACGTGGTACATCAATGCACATGCCATGGTGG CG-3′ (SEQ ID NO: 15), vIRF3(260-270) forward 5′-GATCCGCCACCAT GGAGATGGACATGCTAATGAGGGCGATGTGCGATTAGGC-3′ (SEQ ID NO: 16), vIRF3(260-270) reverse 5′-GGCCGCCTAATCGCACATCGCCCTCATTAGCATGTCCATCTCCATGGTGC G-3′ (SEQ ID NO: 17), vIRF3(270-280) forward 5′-GATCCGCCACCATGGATGAAGACCTCTTTGATCTGCTTGGCATCCCATAG GC-3′ (SEQ ID NO: 18), and vIRF3 270-280 reverse 5′-GGCCGCCTATGGGATGCCAAGCAGATCAAAGAGGTCTTCATCCATGGGT GGCG-3′ (SEQ ID NO: 19).

In addition, PKM2 truncation mutants were generated in a pCMV3 vector with EcoRI and NotI restriction sites using the following primers: PKM2 forward 5′-GCGAATTCCAGCCACCATGTCGAAGCCCATAGTG-3′, PKM2 225 reverse 5′-GCAACTAAGCGGCCGCTTAGTCCTTCTCCCGACACAGC-3′, PKM2 402 reverse 5′-GAATCTTAGCGGCCGCCTTACGCCAGGCGGCGGAGTTC-3′, PKM2 210 forward 5′-GAGAATTCGCGCCACCATGAACCTTCCTGGGGCTGCT-3′, PKM2 107 forward 5′-GAGAATTCGCGCCACCATGCCCGTGCTGTGGCTCTA-3′, PKM2 reverse 5′-GATTCTATGCGGCCGCTCACGGCACAGG AACAAC-3′.

3-3. Establishment and Characterization Analysis of Recombinant KSHV Clones (iSLK-BAC16 Cell Lines)

The iSLK-BAC16-Flag/vIRF3, vIRF3(Δ250-280) and revertants (Rev.) were prepared as follows. First, iSLK cells were infected with each BAC16 DNA using FuGENE HD (Promega). Two days after infection, cells bearing BAC16 were selected using 1 mg/ml hygromycin B, and hygromycin B resistant iSLK-BAC16 cell lines were established after performing the hygromycin B selection for 3 weeks. For the reactivation of recombinant virus, each cell line was treated with 1 μg/ml DOX and 1 mM NaB. After 72 hours of reactivation, cells were harvested through immunoblot analysis using a flag antibody. The culture supernatant of the cells was collected, and filtered through a 0.45 μm SFCA syringe filter. For virion-associated DNA purification, the supernatant was treated with protease K, DNA was extracted with phenol-chloroform, and then KSHV ORF11 specific real-time quantitative PCR was performed. Based on a standard curve using serial dilution of BAC16 DNA, the amount of virus produced by the iSLK-BAC16 cell lines was calculated.

### 3-4. Subcellular Fractionation Assay

Cells were lysed in a fractionation buffer (20 mM HEPES [pH 7.4], 10 mM KCl, 2 mM MgCl₂, 1 mM EDTA, 1 mM EGTA, 1 mM PMSF) and then passed through a 27 G needle (30×). The nuclear pellet was obtained after centrifugation at 720 g for 15 minutes. In addition, the supernatant was removed by centrifugation at 12000 g for 15 minutes (Cytoplasmic fraction). The nuclear pellet was lysed in an NP-40 lysis buffer (150 mM NaCl, 0.5% NP40, 50 mM Tris [pH 8.0]). Both nucleus and cytoplasmic fractions were mixed with a sodium dodecyl sulfate (SDS) loading dye and analyzed by immunoblotting.

### 3-5. Immunoprecipitation and GST-Pull Down

The indicated cells were infected regardless of the presence or absence of combination of plasmids. The cells were collected and lysed in a lysis buffer (150 mM NaCl, 0.5% NP40, 50 mM Tris [pH 8.0]) supplemented with a protease inhibitor cocktail tablet (Thermofisher). Cell lysates were immunoprecipitated against indicated proteins and analyzed by immunoblotting according to methods known in the art.

### 3-6. PKM2 Cross-Linking and Gradient PAGE Gel Running

293T cells were infected with indicated combination of plasmids and harvested 48 hours after infection. The cells were lysed with a NP40 lysis buffer supplemented with a protease inhibitor (150 mM NaCl, 0.5% NP40, 50 mM pH 8.0 Tris-HCl) and treated with 0.1% glutaraldehyde for 4 minutes at room temperature. Thereafter, gradient SDS-PAGE was performed with NuPAGE 4-12% Bis-Tris gel (Invitrogen) and analyzed by immunoblotting.

### 3-7. Pyruvate Kinase Activity Assay

Pyruvate kinase activity assay was performed with a Pyruvate Kinase Assay Kit (Sigma Aldrich) according to manufacturer's instructions. Briefly, LECs expressing a pCDH vector, vIRF3, and vIRF3(Δ250-280), respectively were harvested and washed twice with 1×PBS. The cells were suspended in 50 μl of a pyruvate kinase assay buffer and incubated for 15 minutes. Cell debris was removed by centrifugation at 13,000 rpm for 15 minutes. The supernatant was transferred to a 96-well plate and added with a kinase assay mixture. Absorbance was measured at 570 nm wavelength using a Varioskan Lux microplate reader (Thermo scientific).

### 3-8. Acetyl-CoA Assay

Acetyl-CoA assay was performed using an acetyl-CoA assay kit (Sigma Aldrich) according to manufacturer's instructions. Briefly, LEC cell lines were harvested and lysed with 50 μl of an acetyl-CoA assay buffer. Lysates were deproteinized with perchloric acid and neutralized with KOH. The supernatants were transferred to a 96-well plate and mixed with a reaction mixture, and then the fluorescence intensity was measured at λex=535 nm and λem=587 nm.

### 3-9. Lactate and Adenosine Triphosphate (ATP) Assay

Lactate and ATP assay were performed with Lactate-Glo and CellTiter-Glo (Promega) according to manufacturer's instructions. Briefly, cells were seeded on a 96-well plate and incubated for 24 hours. After culture, the culture medium and cells were obtained, respectively. The culture medium was mixed with a lactate detection mixture and incubated in the 96-well plate for 1 hour. The cells were washed and suspended in 1×DPBS and then added with an equal amount of CellTiter-Glo. After incubating the cell lysate for 10 minutes, the cell lysate was transferred to a 96-well plate. A relative luminescence unit (RLU) of the lactate and ATP assay mixture was measured by a Varioskan Lux microplate reader.

### 3-10. Metabolites Extraction and LC/MS-Based Metabolomics Analysis

LEC-E6/E7 cell lines bearing each pCDH, V5/vIRF3, or V5/vIRF3(Δ250-280) were seeded on a 100 mm cell culture plate. After 48 hours, cells were harvested using cold 80% methanol. The harvested cells were homogenized with 0.1 mm zirconia/silica beads using BeadRuptor Elite (Omni). The beads and cell debris were removed by centrifugation at 13,000 rpm for 10 minutes. The supernatant was transferred into a cryotube and lyophilized with liquid N₂. The samples were stored at −80° C. before analysis by LC/MS. Metabolites were extracted therefrom according to methods known in the art, and the extracted metabolites were separated on a Cogent Diamond Hybride TYPE-C column (gradient 3) and the LC/MS analysis of the metabolites was performed using a mobile phase consisted of the following (using 0.2% formic acid). The gradient used and time conditions were as follows: 0-2 min, 85% sol B; 3-5 min, 80% sol B; 6-7 min, 75% sol B; 8-9 min, 70% sol B; 10-11.1 min, 50% sol B; 11.1-14 min, 20% sol B; 14.1-24 min, 5% sol B followed by a 10-min re-equilibration period at 85% sol B at a flow rate of 0.4 mL/min. At this time, an Agilent Accurate Mass 6230 time of flight mass spectrometer coupled with an Agilent 1290 liquid chromatography (LC) system was used. Detected ions were regarded as metabolites on the basis of unique accurate mass-retention time identifiers for masses exhibiting the expected distribution of accompanying isotopes. Metabolite identities were searched using a mass tolerance of less than 0.005 Da, and the rich extracted metabolite ion intensities were extracted using Profinder B.08.00 and Agilent Qualitative Analysis B.07.00 (Agilent Technologies).

### 3-11. Immunofluorescence Assay

Cells seeded on a glass disc were fixed with 4% paraformaldehyde (Sigma) and permeabilized with 0.1% Triton X-100 (Sigma). Primary antibodies targeting indicated proteins were diluted to a concentration recommended in the manufacturer and added to the cells. Secondary antibodies conjugated with FITC, Rhodamine, or Alexa Fluor®568 were diluted and added to the cells. After staining, a mounting solution containing DAPI (Vector Laboratories) was dropped onto a slide glass and the disc with the cells was placed on the mounting solution. Prepared slides were analyzed with confocal laser scanning microscope LSM-700 (Carl Zeiss).

### 3-12. Transwell Cell Migration Assay

Transwell cell migration assay was performed using a 24-well transwell insert (Corning) with 8 μm-pore sizes. A cell culture medium containing 10% FBS was added to each well of the 24-well plate and the transwell insert was disposed in each well. LEC cell lines were collected and re-suspended in a serum-free culture medium. A cell suspension was added into the transwell insert and incubated for 16 hours. After incubation, the cells were fixed and stained with a 0.05% crystal violet solution containing 1% formaldehyde and 25% methanol. The non-migrated cells on the top-side of the transwell were removed with a cotton swab and the transwell was washed with 1×PBS. The migrated cells on the bottom-side of the transwell were counted and imaged with the EVOS-M500 imaging system (Thermo Scientific).

### 3-13. 3D Spheroid Invasion Assay

LEC cell lines were seeded on a 96 well ultra-low attachment (ULA) plate (Corning) and incubated for 48 hours. Spheroids were transferred onto a 3 mg/ml matrigel (Corning) layer in the 96 well plate and incubated for 2 hours at 37° C. After incubation, the matrigel was added on the wells to make an upper layer. The culture medium was added onto the upper layer and incubated for 3 days. The matrigel matrixes containing spheroids were fixed with acetone/methanol (1:1) and blocked with a blocking buffer (15% heat-inactivated PBS, 0.3% Triton X-100 (1×PBS)). Thereafter, the spheroids were stained with indicated primary and secondary antibodies and followed by z-stack analysis with a confocal laser scanning microscope LSM-700.

### 3-14. Chorioallantoic Membrane (CAM) Invasion Assay

The chick fertilized eggs were incubated at 37.5° C. with 70% humidified atmosphere in a Rcom D-50 incubator (Autoelex). After embryonic day (ED) 5, a 1.5 cm square hole was opened on the eggshell and sealed with a sterile parafilm tape. Embryos were monitored by daily candling. On ED 10, sterile silicone rings were placed onto the CAM of the embryo and added with indicated cells, and then the cells were seeded. 0.2 mg of TAT (a control peptide containing only a TAT sequence of HIV-1 (synthesized by Bionics)) or TAT-VDP according to the present invention (vRIF3-derived aa250-260 peptide bound with a TAT sequence (synthesized by Bionics)) was treated at 24 and 48 hours after cell seeding. Approximately 1 cm² of CAM around the silicone ring was harvested and fixed with 4% paraformaldehyde at 4° C. for 24 hours. Thereafter, samples were treated for immunohistochemistry and Hematoxyling and Eosin (H&E) staining.

### 3-15. Xenograft Model

LECs (LEC-E6/E7/V5/vIRF3) expressing vIRF3 and immortalized through expression of E6/E7 were washed with PBS and injected subcutaneously into 7-week-old female avascular NCr-nu/nu mice (Koatech). 15 mice were used in each experimental group. The TAT or TAT-VDP peptide was dissolved in 1×DPBS and administered subcutaneously to the cell injection site at a dose of 0.2 mg per dose, twice a week, 3 days after cell inoculation. Tumor growth inhibition was monitored by comparing body weights and tumor volumes between TAT and TAT-VDP treated mice daily for 30 days. The tumor volume was calculated using the following Equation 1.

$$Tumor volume = 1 / 2 ( length \times {width}^{2} ) \qquad [ Equation 1 ]$$

After monitoring for 30 days, TAT-VDP was administered to the TAT-treated experimental group to evaluate tumor regression. All data were analyzed using GraphPad Prism software and a two-tailed unpaired t-test.

### 3-16. Immunohistochemistry

After euthanizing the mice, organs and tumors were dissected and immediately fixed in 4% paraformaldehyde. After 24 hours of fixation, immunohistochemistry was performed using tissue samples by the Genoss (Suwon, Korea).

### 3-17. Statistics

Statistical analysis was performed using GraphPad Prism software. P-values were calculated using a Student's t-test or one-way ANOVA test. P<0.05 indicated statistical significance, and data were expressed as mean±SD.

### Example 1. Confirmation of Interaction Between KSHV vIRF3 and PKM2 in LEC

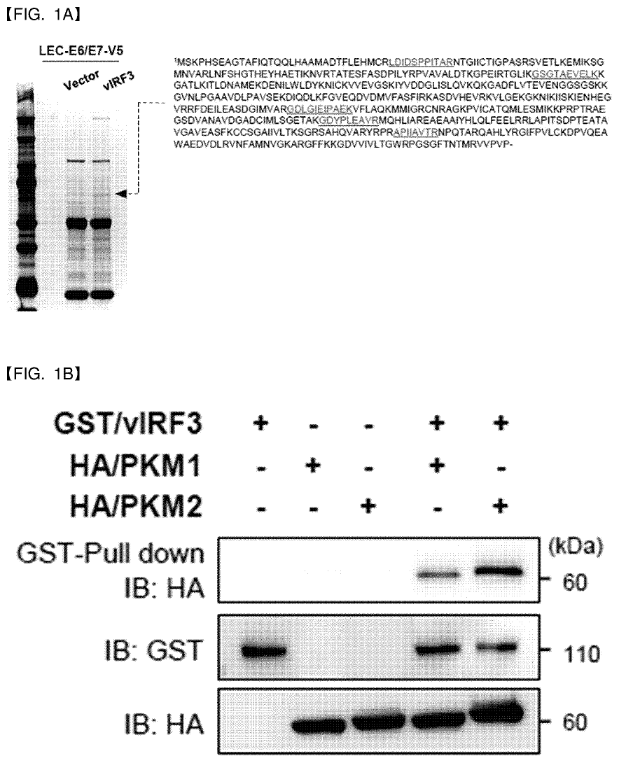

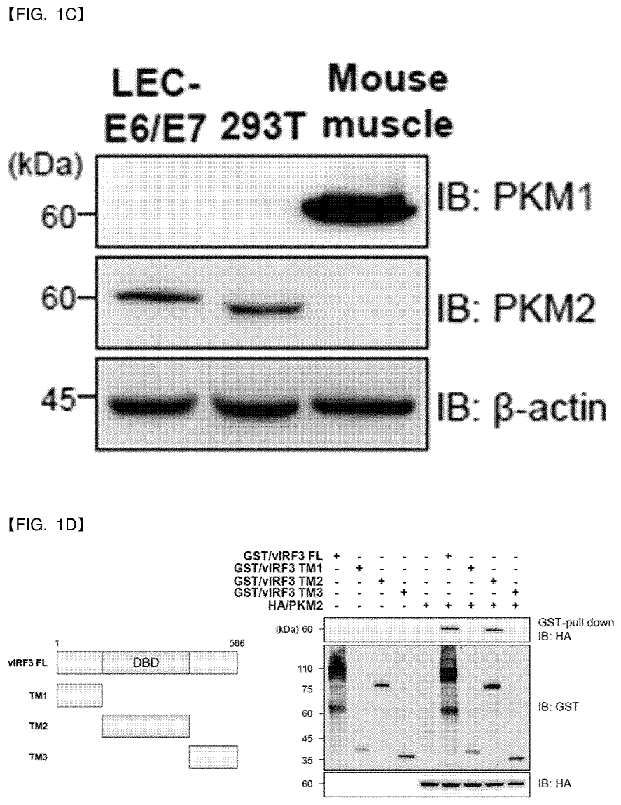

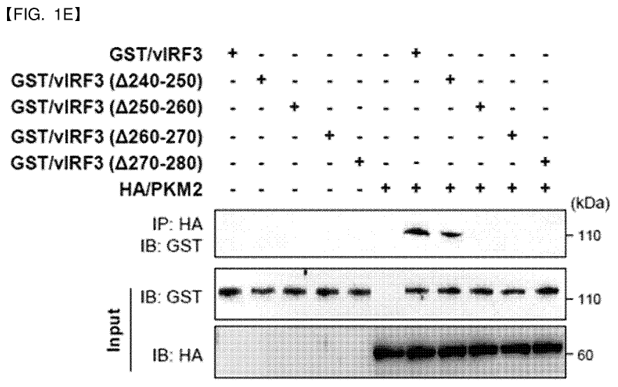

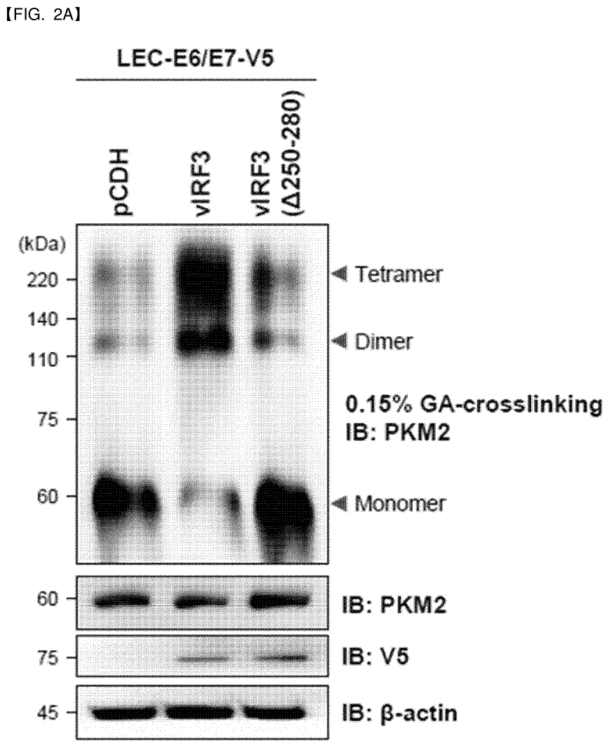

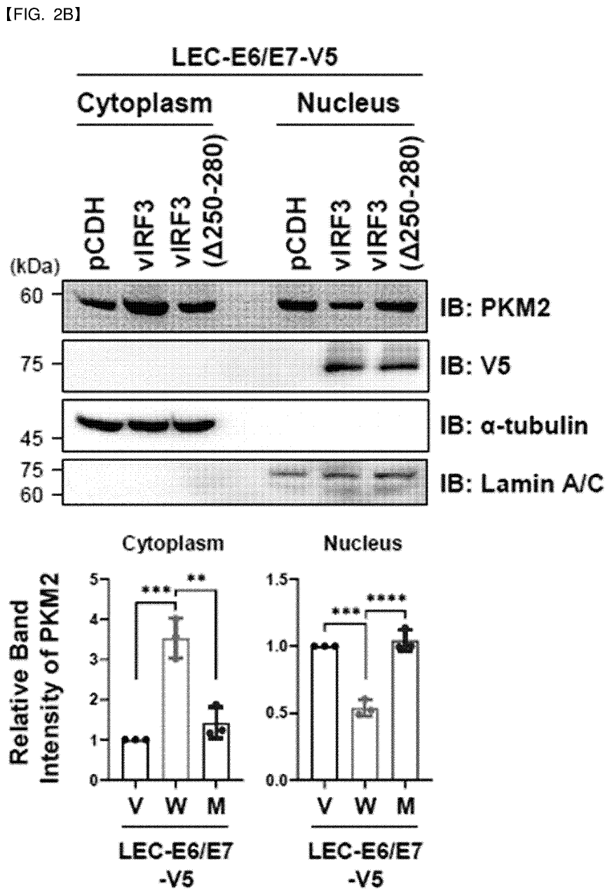

To identify a KSHV vIRF3 interaction network in lymphatic endothelial cells (LECs), a mass spectrometry analysis was performed using V5-tagged vIRF3 expressing LECs. As a result, polypeptide with a molecular mass of ˜60 kDa was identified as pyruvate kinas M (PKM) (FIG. 1A). PKM genes encoded PKM1 and PKM2, which were produced by alternative splicing. Both PKMs catalyzed the final step of glycolysis to convert phosphoenolpyruvate (PEP) to pyruvate and generate ATP. PKM1 is expressed in terminally differentiated tissues such as muscle and brain, while PKM2 is mainly expressed in highly proliferative cells, such as stem cells, embryonic cells, and tumor cells. In addition, PKM2 was expressed only in LECs, whereas PKM1 was not detected at the transcriptional level in LECs (FIG. 1B). As a result, vIRF3 interacted with PKM2 through immunoprecipitation (FIG. 1C). Binding of a vIRF3 N-terminal domain (1-150; TM1), a central domain (151-430; TM2), a C-terminal domain (430-566; TM3) and wild-type vIRF3 was confirmed through a precipitation method, and as a result, binding of the vIRF3 wild-type and the TM2 (151-430) domain was confirmed (FIG. 1D). Furthermore, as a detailed binding assay result between vIRF3 and PKM2, it was indicated that vIRF3₂₅₀₋₂₈₀ was specifically involved with PKM2₂₁₀₋₄₀₂ (FIGS. 1D and 1E). In addition, LEC lines which ectopically expressed V5-tagged vIRF3 or vIRF3 (Δ250-280), were generated. As a result analyzed by immunoprecipitation, it was confirmed that vIRF3 interacted with endogenous PKM2, but mutants in which vIRF3-derived fragments (250-260, 260-270, 270-280) were deleted did not bind to PKM2 (FIG. 1E). Through the above results, it was confirmed once again that a 250 to 280aa region of vIRF3 was a region required for PKM2 binding.

### Example 2. Confirmation of Enhanced Pyruvate Kinase (PK) Activity by KSHV vIRF3-PKM2 Interaction

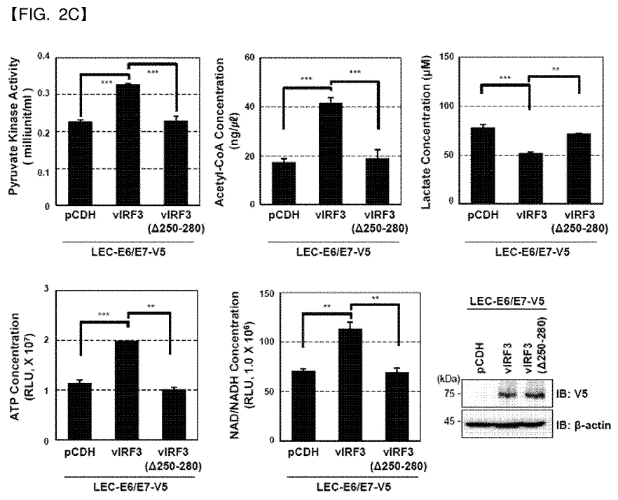

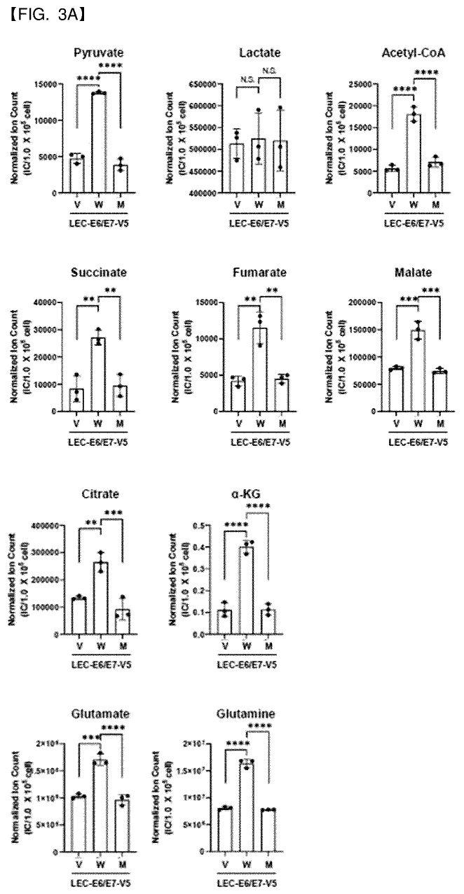

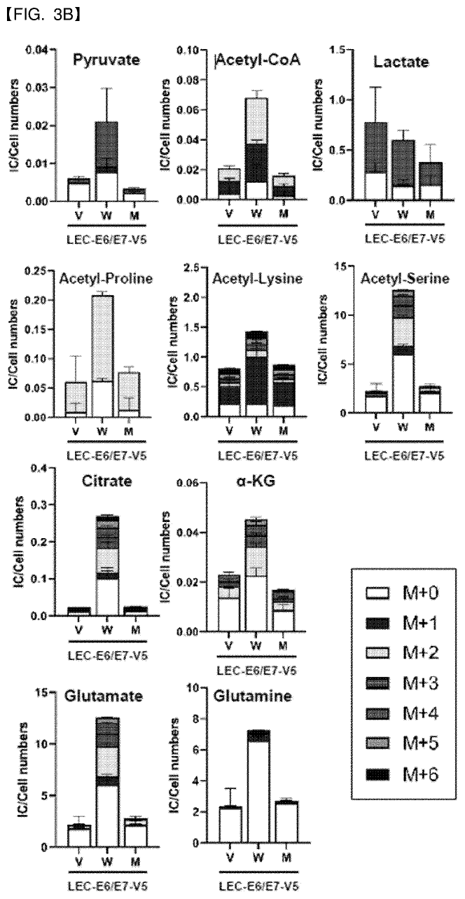

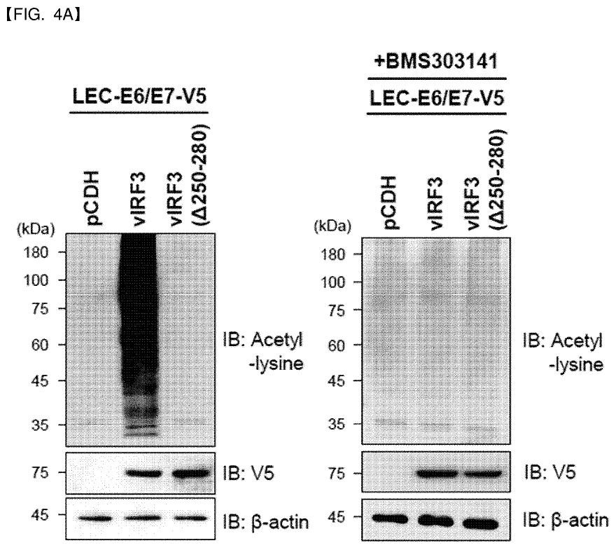

It is known that the biological properties of PKM2 between dimeric and tetrameric forms are significantly different. It is known that the tetrameric form mainly acts as PK and promotes glycolysis and oxidative phosphorylation, while the dimeric form PKM2 serves as a transcriptional co-factor in the nucleus. As a result of investigating whether vIRF3-PKM2 interaction influenced the allosteric state of PKM2, when vIRF3 was expressed in LECs, it was found that vIRF3 dramatically enhanced the tetramerization of PKM2, while vIRF3(Δ250-280) was not influenced (FIG. 2A). Further, vIRF3 induced the accumulation of PKM2 in the cytoplasm, and downregulated the PKM2 expression even in the nucleus (FIG. 2B). To further confirm the role of vIRF3-mediated PKM2 tetramerization, the PK enzymatic activity was measured. As a result, the presence of KSHV vIRF3 increased the PK activity-related acetyl-CoA concentration, while the lactate concentration was decreased under the same condition (FIG. 2C). Meanwhile, it was confirmed that vIRF3(Δ250-280) did not induced changes in PK enzyme activity and its related states unlike vIRF3.

### Example 3. Confirmation of KSHV vIRF3-PKM2 Interaction in TCA Cycle Metabolites

To study the impact of full-length vIRF3 expression on LEC cellular metabolism, LEC-vector, LEC-V5/vIRF3, and LEC-V5/vIRF3(Δ250-280) were cultured under 2D monolayer conditions. vIRF3-induced LEC metabolites were extracted and the changes thereof were analyzed using Metaboanalyst v5.0. At this time, the LEC metabolites were analyzed with focus on TCA cycle intermediates. First, it was found that pyruvate and acetyl-CoA levels rich in LEC-vIRF3 were significantly increased compared to a vector alone or vIRF3(Δ250-280) mutants in LEC-vIRF3. The acetyl-CoA was not lactate. The enhanced acetyl-CoA level was preferentially used as a substrate of TCA cycle intermediates identified by the increased biosynthesis of all TCA cycle intermediates including citrate, alpha-KG, succinate, fumarate, and succinate. Interestingly, accumulated glutamate and glutamine were also observed. To clarify the origin of TCA cycle intermediates between glycolytic flux and glutaminolysis, a 13C isotope tracing experiment was performed using fully 13C labeled ([U-13C]) glucose as a single carbon source. As expected, the enhanced pyruvate is composed primarily of M+3 in LECs overexpressing full-length vIRF3 instead of vIRF(Δ250-280) by the catalytic activity of triose-phosphate isomerase. Then, the pyruvate serves as a substrate of PKM2 to produce acetyl-CoA, mainly in a M+1 or M+2 isotope. Since the acetyl-CoA is used directly as a substrate for TCA cycle intermediates, M+2 is a major isotopic component of all TCA cycle intermediates, which supports the hypothesis that the enhanced biosynthesis of vIRF3-mediated acetyl-CoA is mediated by the increased PKM2 catalyst activity, and the acetyl-CoA serves as a substrate for the TCA cycle intermediate biosynthesis. In addition, it was confirmed that enhanced glutamate biosynthesis was primarily mediated by alpha-KG. It was also observed that the levels of acetylated proline, lysine, and serine in LEC-vIRF3 rather than LEC-vIRF3(Δ250-280) were increased. The 13C isotope tracing experiment also confirmed that acetyl-CoA accumulated by vIRF3-PKM2 also serves as a substrate to acetylate amino acids.

### Example 4. Confirmation of Influence of KSHV vIRF3-PKM2 on Intracelluar Acetyl-CoA Derived from Pyruvate Catabolism

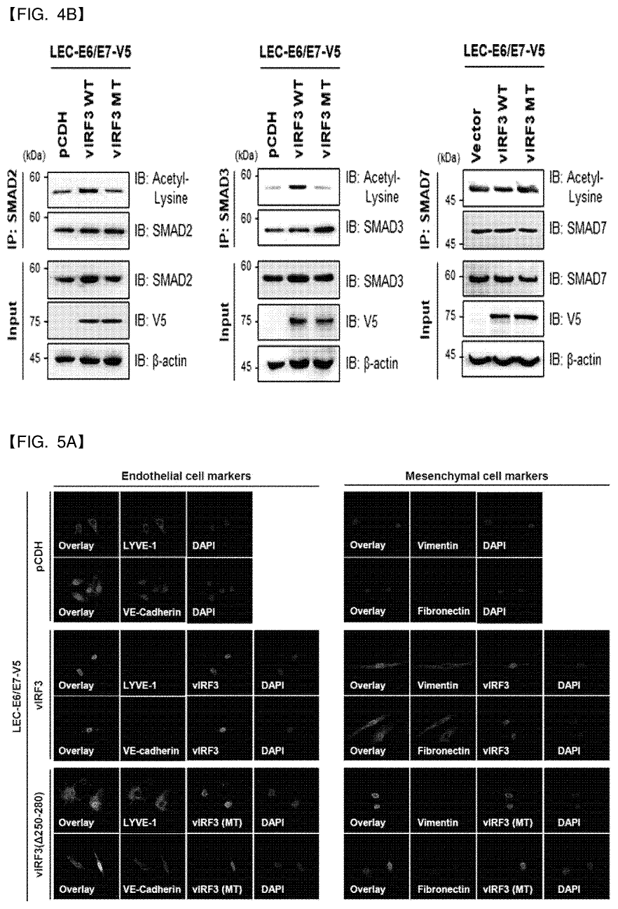

Acetyl-CoA is a crucial metabolite that provides acetyl groups for acetylation reaction of proteins including histone. To better understand the importance of acetyl-CoA upregulation by vIRF3 in LEC, first, it was confirmed whether vIRF3 may increase the overall acetylation level in LEC. To this end, cell lysates were analyzed by immunoblotting (IB) using an anti-acetyl-lysine antibody. Consistently with the metabolomics analysis, constitutive vIRF3 expression dramatically increased the entire acetylation level in LEC (FIG. 4A). Especially, cytosolic acetyl-CoA, which was involved in lysine acetylation of protein, regulating the activity, stability, or localization of many cell proteins is primarily generated by the translocation of citrate from the mitochondria to the cytoplasm via ATP-citrate lyase (ACLY). Therefore, three LEC stable cell lines encoding expression vectors of pCDH, V5-vIRF3, and V5-vIRF3(Δ250-280) were treated with an ACLY inhibitor BMS303141 and then analyzed by IB with an anti-acetyl-lysine antibody. As a result, since the increase of total acetylation levels was not confirmed, the increased acetylation was caused from TCA cycle-derived citrate (FIG. 4B).

Meanwhile, increased TGF-β signaling has been recognized as a major underlying mechanism of EndMT-related diseases. Moreover, it was reported that alteration in intracellular acetyl-CoA levels and SMAD7 signaling in blood endothelial cells (BECs) are critical for EndMT. Accordingly, to investigate whether a high level of vIRF3-PKM2 interaction-mediated acetyl-CoA affected TGF-β signaling, pull down assay using specific antibodies against a TGF-β downstream effector SMAD and IB using an acetyl-lysine antibody were conducted. As a result, the vIRF3 expression increased the acetylation of SMAD2 and SMAD3, but did not increase SMAD7 as an inhibitory SMAD (FIG. 4B). Therefore, it was found that the high level of acetyl-CoA associated with vIRF3-PKM2 increased endogenous TGF-β signaling.

### Example 5. Confirmation of Effect by vIRF3-Mediated Acetylation of SMAD2 and SMAD3

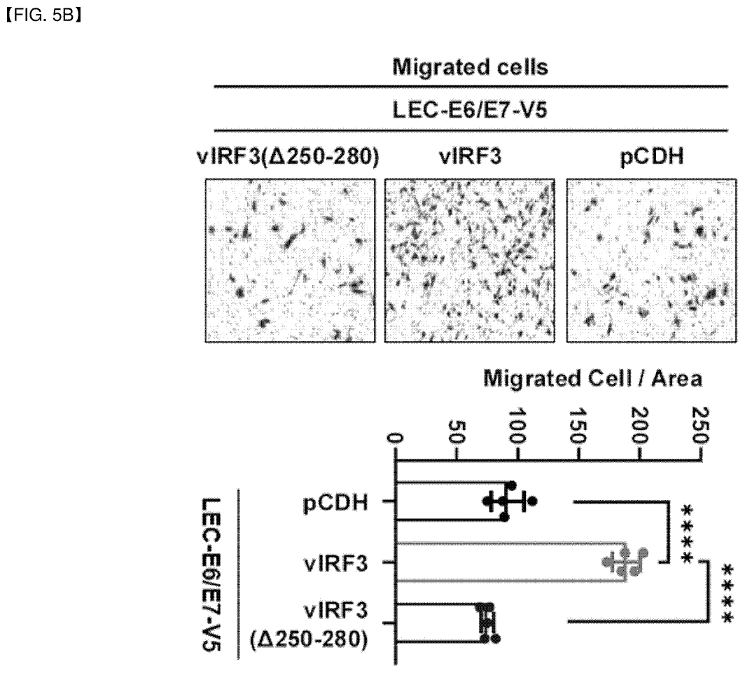

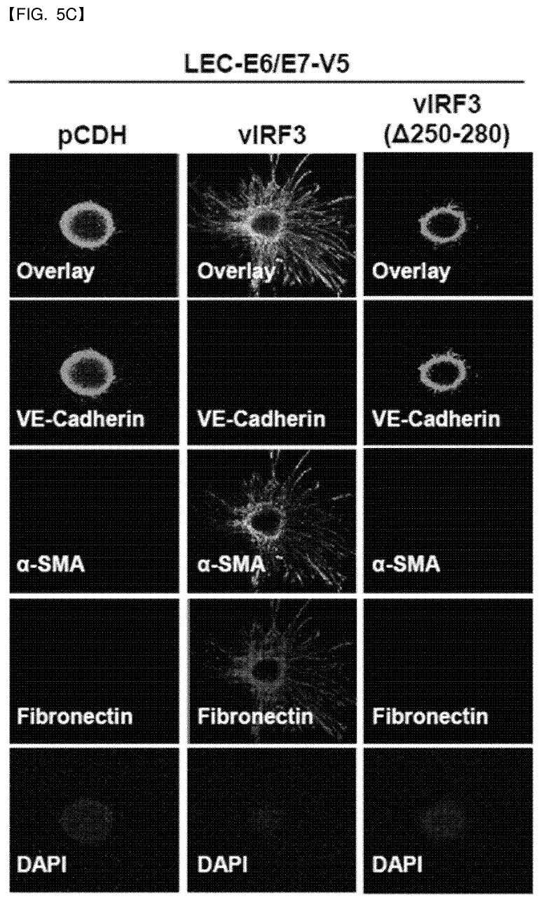

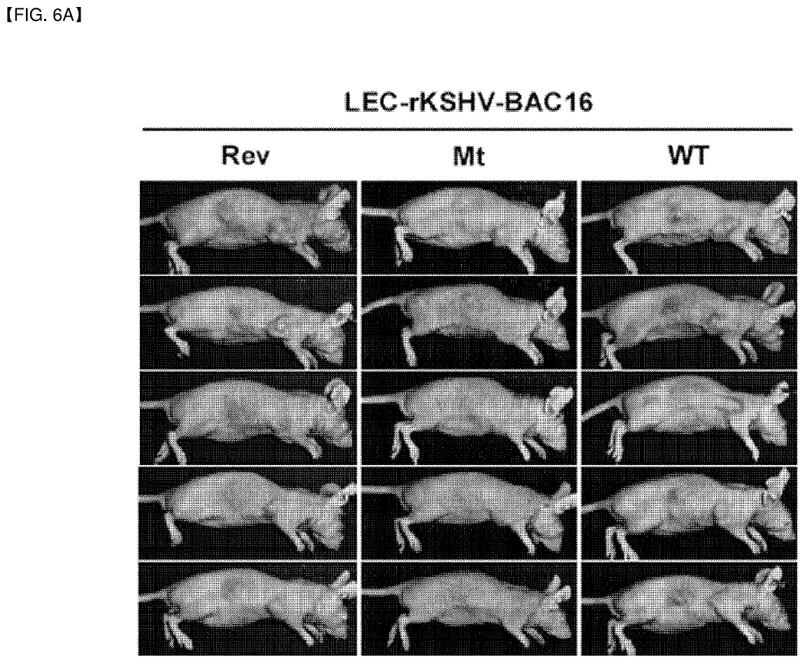

Based on the fact that both SMAD2 and SMAD3-mediated TGF-β signaling activation was required to induce EndMT, next, it was determined whether LEC-vIRF3 could express typical mesenchymal markers while inhibiting typical endothelial cell markers. As a result, it was confirmed that LYVE-1 and VE-cadherin among endothelial markers were significantly decreased by vIRF3. In contrast, upregulated mesenchymal markers (vimentin, fibronectin, N-cadherin, and α-smooth muscle actin (α-SMA) were decreased. However, LEC-vIRF3(Δ250-280) did not downregulate the endothelial markers without interacting with PKM2 and did not upregulate the mesenchymal markers (FIG. 5A).

Since invasiveness was increased as the major characteristics of mesenchymal cells were increased, first, the potential functional contribution of migration by vIRF3 was investigated using Boyden chamber assay. Three LEC stable cell lines expressing pCDH, V5-vIRF3 and V5-vIRF3(Δ250-280) were plated in an upper chamber of 8 μm-pore size transwells, and migration was measured after 16 hours. As a result, vIRF3-expressing LECs showed increased migration behavior (2-fold), whereas vIRF3 expression (Δ250-280) was not different from a vector cell line (FIG. 5B).

To further investigate the invasion capability of LEC-vIRF3, a 3D spheroid invasion assay was performed by embedding multicellular tumorigenic spheroids into 3D Matrigel using the three LEC stable cell lines. To analyze invasiveness, a cell-covered area was divided into three distinct subregions: a core, a middle, and an edge. The core is a central region where cells are densely aggregated, the middle region is a region where cells are somewhat loosely aggregated, and the edge is a region where cells invade the surrounding region. Consistent with the migration analysis data, only 20% of the core region was detected, while both the middle and edge regions were significantly increased upon vIRF3 expression. In contrast, the expression of the vector and V5-vIRF3(Δ250-280) showed the edge region of the mediator. In addition, as the confocal microscopy analysis result, in contrast to the vector and V5-vIRF3 (Δ250-280), invasion of vIRF3-stimulating spheroids expressed mesenchymal markers (αSM, fibronectin), but an EC marker, VE-cadherin showed a very faint signal (FIG. 5C).

Through this, it was confirmed that mesenchymal reprogramming of LECs induced by vIRF3-PKM2 interaction induced migration and invasion that may play a critical role in KSHV-associated tumorigenesis, and since vIRF3(Δ250-280) did not induce the migration and invasion, it was confirmed that it is possible to suppress KSHV-related tumor formation using the same.

### Example 6. Confirmation of Effect of KSHV vIRF3 and PKM2 Interaction in Xenograft Model

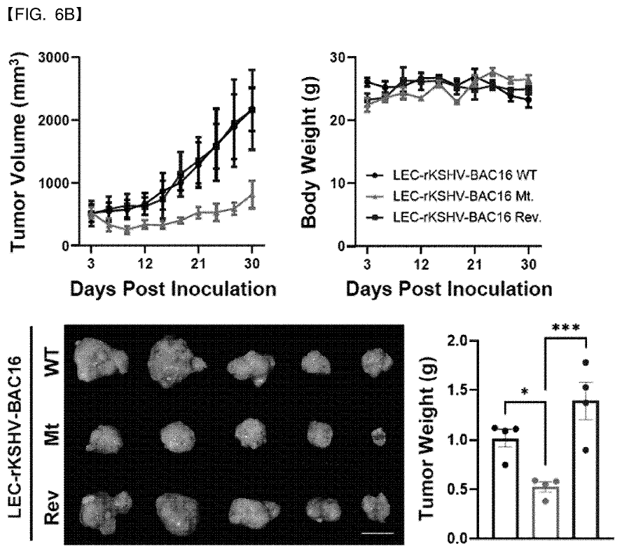

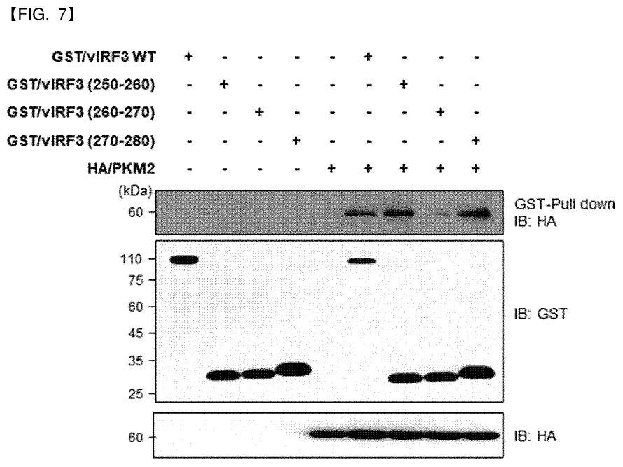

To investigate the role of EndMT through vIRF3-PKM2 interaction in KSHV-associated pathogenesis, first, a subcutaneous xenograft experiment was performed with LECs infected with WT-, MT-, or Rev-BAC16 KSHV, and then tumor growth, volume, weight, and body weight were determined. As a result, both LECs infected with WT- and Rev-BAC16 KSHV effectively induced tumor initiation and promotion compared to LEC infected with MT-BAC16 KSHV (FIGS. 6A and 6B). To investigate whether vIRF3-PKM2-associated EndMT could induce secondary tumors observed in cancer cells in other internal organs, mice were sacrificed 30 days after transplantation of three different KSHV-infected cells. Interestingly, consistent with the tumor promotion results shown in FIGS. 6A and 6B, morphologically abnormal organs were found in both WT- and Rev-BAC16 KSHV, and GFP expression indicating the presence of KSHV genome was detected in several other organs, including stomach, spleen, and liver.

### Example 7. Confirmation of vIRF3-Mediated PKM2 Function Inhibitory Effect of KSHV vIRF3-Derived Peptide

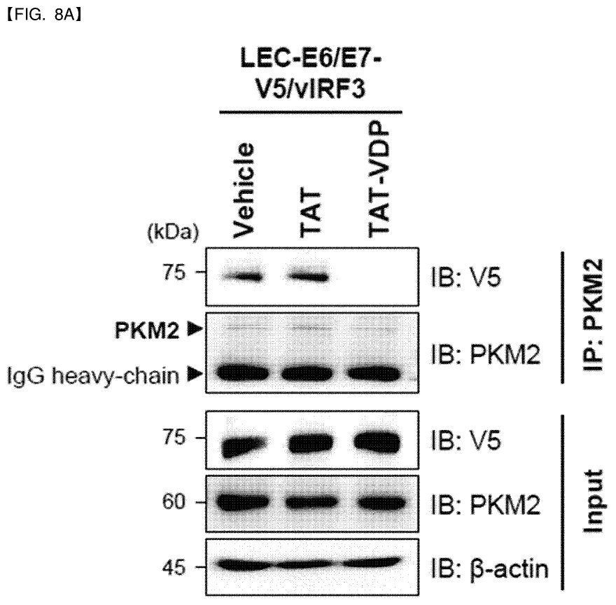

Based on the results, it was confirmed that an amino acid region 250-280 of vIRF3 played an important role in the interaction with PKM2, and as a result of confirming whether its fragments vIRF3(250-260), vIRF3(260-270), and vIRF3(270-280) also bound to PKM2, it was confirmed that vIRF3(250-260), vIRF3(260-270), and vIRF3(270-280) also mutual-bound to PKM2 (FIG. 7).

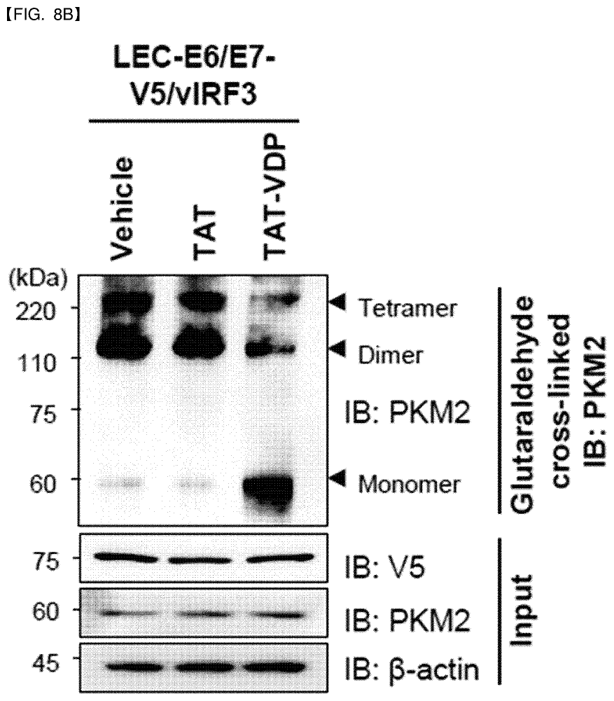

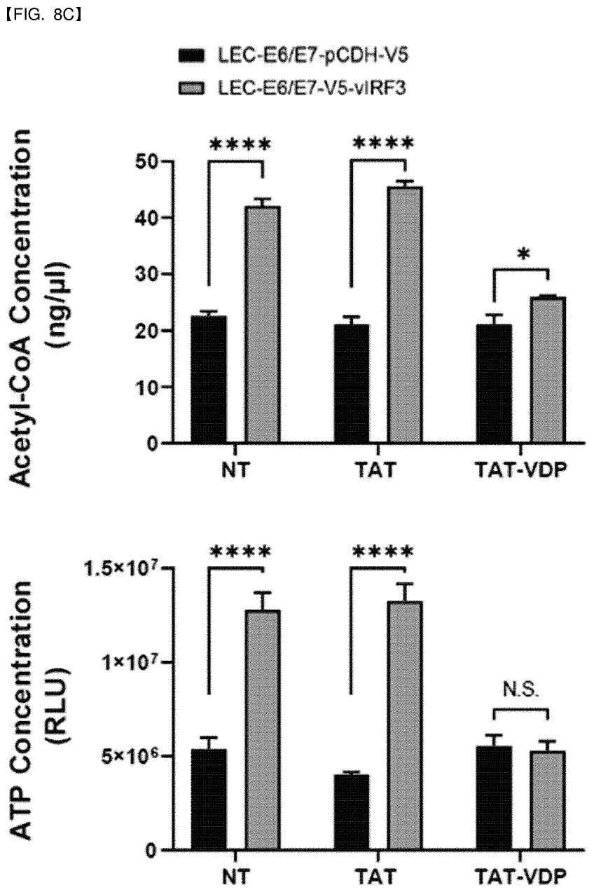

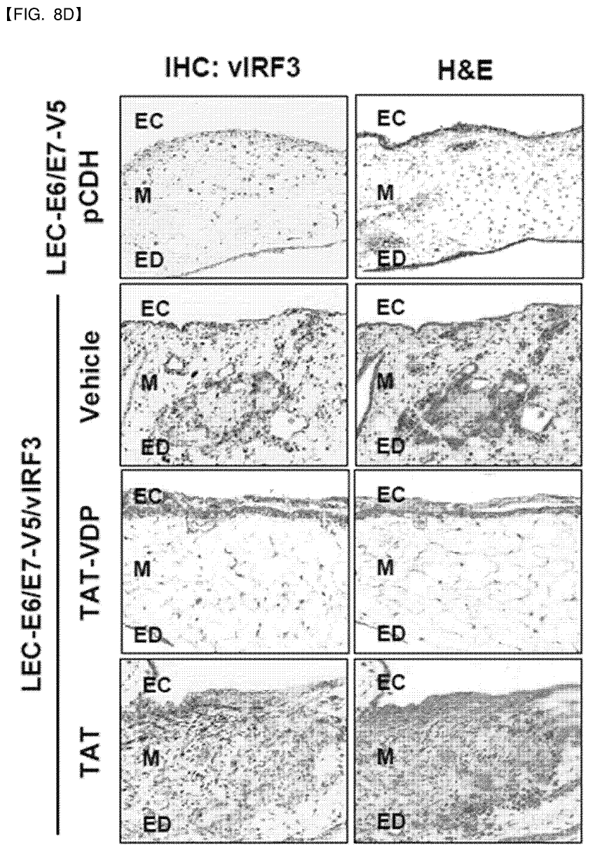

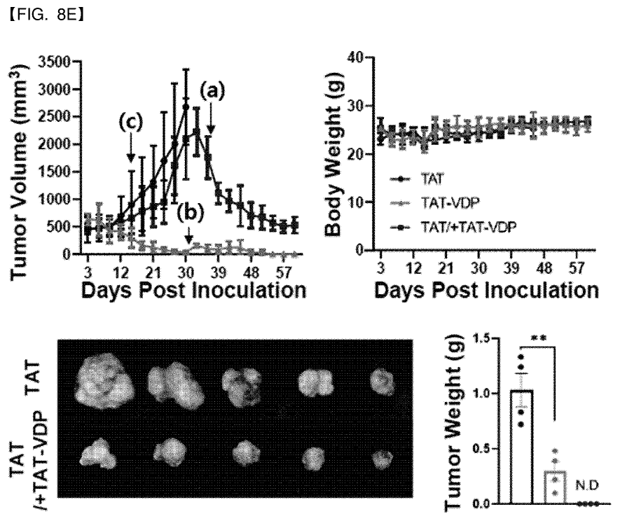

In addition, the effect of A vIRF3-derived aa250-260 peptide (hereinafter referred to as ‘VDP’) on a pyruvate metabolic cycle was confirmed. In this regard, VDP was fused to a HIV-1 TAT protein transduction domain for intracellular delivery. Thereafter, LEC-V5-vIRF3 or LEC-vector cells were treated with TAT or TAT-VDP and then several assays were performed to measure PK activity and downstream metabolite accumulation. As a comparative kinetic assay result, it was confirmed that VDP effectively inhibited PK activity, acetyl-CoA concentration, and ATP concentration upregulated by vIRF3 expression in LEC, but showed no effect on LEC-vector cells (FIG. 8C). Thereafter, it was investigated whether VDP may reduce a high level of vIRF3-mediated PK activity through vIRF3 binding competition. To this end, LEC-V5-vIRF3 was treated with TAT or TAT-VDP, and then immunoprecipitation was performed with an anti-PKM2 antibody. In contrast to TAT treatment, VDP efficiently blocked the vIRF3-PKM2 interaction (FIG. 8A). Moreover, as expected, TAT-VDP sufficiently prevented a vIRF3-mediated tetrameric form of PKM2 by competing with vIRF3 for interaction with PKM2 (FIG. 8B).

In addition, to evaluate anti-invasive activity of VDP, LEC-V5-vIRF3 was injected into a chick chorioallantoic membrane (CAM). After establishment of invasion in Ovo, TAT or TAT-VDP was treated, and then the CAM was harvested for immunohistochemistry (IHC). The histological structure of the CAM includes three major germ layers of ectoderm, mesoderm, and endoderm. As a result, LEC-V5-vIRF3 was induced to be invaded into the mesoderm layer of CAM, but the LEC-vector detected only the ectoderm layer of CAM (FIG. 8D). Particularly, VDP-treated LEC-V5-vIRF3 showed minimal invasion into an ectodermal group, whereas the TAT treatment was not affected on vIRF3-mediated invasion ability, which suggests that VDP effectively inhibited the invasive activity (FIG. 8D). To further confirm the antitumor activity of VDP, NOD/SCID xenograft mice were used with subcutaneous injection of recombinant KSHV-infected LECs. After injecting the LECs infected with KSHV, the mice were challenged with 0.2 mg (equivalent to ˜μM) of each peptide twice per week for 30 days. As a result treated with TAT-VDP, tumor formation was remarkably inhibited unlike a TAT-treated experimental group (FIG. 8E). Therefore, it was confirmed that the vRIF3-derived peptide according to the present invention had excellent antitumor activity.

In summary, the Kaposi's sarcoma-associated herpesvirus (KSHV) vIRF3-derived peptide according to the present invention has the effects of inhibiting the activity of PKM2, reducing the size and volume of tumors, and inhibiting the migration and invasion of cancer cells, and thus can be advantageously utilized for the prevention, alleviation or treatment of cancer, or for inhibiting the metastasis of cancer cells.

1. A peptide derived from viral interferon regulatory factor 3 (vIRF3) or a fragment thereof, consisting of an amino acid sequence represented by SEQ ID NO: 1.

2. The vIRF3-derived peptide or the fragment thereof of claim 1, wherein the vIRF3-derived peptide consisting of the amino acid sequence represented by SEQ ID NO: 1 is vIRF3 aa 250-280 consisting of the amino acid sequence represented by SEQ ID NO: 2.

3. The vIRF3-derived peptide or the fragment thereof of claim 1, wherein the fragment is a fragment of a vIRF3-derived peptide consisting of one amino acid sequence selected from the group consisting of amino acid sequences represented by SEQ ID NOs: 3 to 5.

4. The vIRF3-derived peptide or the fragment thereof of claim 1, wherein the vIRF3 is Kaposi's sarcoma-associated herpesvirus (KSHV) vIRF3.

5. The vIRF3-derived peptide or the fragment thereof of claim 1, wherein a cell permeable domain is further linked to the vIRF3-derived peptide.

6. The vIRF3-derived peptide or the fragment thereof of claim 5, wherein the cell permeable domain is an HIV TAT peptide.

7. The vIRF3-derived peptide or the fragment thereof of claim 6, wherein the vIRF3-derived peptide further bound to the cell permeable domain consists of an amino acid sequence represented by SEQ ID NO: 6.

8. The vIRF3-derived peptide or the fragment thereof of claim 1, wherein the vIRF3-derived peptide consisting of the amino acid sequence represented by SEQ ID NO: 1 or the fragment thereof inhibits the activity of pyruvate kinase M2 (PKM2).

9. A pharmaceutical composition for preventing or treating cancer, comprising a vIRF3-derived peptide of claim 1, a fragment thereof, or a polynucleotide encoding the same.

10. The pharmaceutical composition for preventing or treating cancer of claim 9, wherein the cancer is cancer that overexpresses pyruvate kinase M2 (PKM2).

11. The pharmaceutical composition for preventing or treating cancer of claim 10, wherein the cancer is at least one selected from the group consisting of lung cancer, prostate cancer, colon cancer, breast cancer, lymphoma, kidney cancer, pancreatic cancer, liver cancer, ovarian cancer, and gallbladder cancer.

12. A method for preventing or treating cancer in a subject other than humans, comprising administering to a subject other than humans a pharmaceutical composition according to claim 9.

13. A pharmaceutical composition for inhibiting migration or metastasis of cancer, comprising a vIRF3-derived peptide of claim 1, a fragment thereof, or a polynucleotide encoding the same.

14. A method for screening a cancer therapeutic agent, comprising:
(a) contacting a candidate substance with a sample containing PMK2 consisting of an amino acid sequence represented by SEQ ID NO: 7;
(b) comparing a physical interaction of the candidate substance with the PKM2 domain with a physical interaction of the vIRF3-derived peptide or fragment thereof according to claim 1; and
(c) selecting the candidate substance as a cancer therapeutic agent when the physical interaction of the candidate substance is similar to or greater than the physical interaction of the vIRF3-derived peptide or the fragment thereof.

15. A quasi-drug for preventing or alleviating cancer comprising a vIRF3-derived peptide of claim 1, a fragment thereof, or a polynucleotide encoding the same.

16. A food composition for preventing or alleviating cancer comprising a vIRF3-derived peptide of claim 1, a fragment thereof, or a polynucleotide encoding the same.

17. A health functional food composition for preventing or alleviating cancer comprising a vIRF3-derived peptide of claim 1, a fragment thereof, or a polynucleotide encoding the same.

18. A method for treating cancer, comprising treating a subject with a peptide derived from viral interferon regulatory factor 3 (vIRF3) consisting of an amino acid sequence represented by SEQ ID NO: 1, a fragment thereof, or a polynucleotide encoding the same.

19. The method for treating cancer of claim 18, wherein the cancer is cancer that expresses PKM2.

20. A method for inhibiting metastasis of cancer, comprising treating a subject with a peptide derived from viral interferon regulatory factor 3 (vIRF3) consisting of an amino acid sequence represented by SEQ ID NO: 1, a fragment thereof, or a polynucleotide encoding the same.


SEQUENCE LISTING (19 sequences)

SEQ ID NO: 1 - protein, 31 aa, synthetic construct
ACALMYHVGQEMDMLMRAMCDEDLFDLLGIP

SEQ ID NO: 2 - protein, 1701 aa, synthetic construct
ATGGCGGGACGCAGGCTTACCTGGATTTCTGAGTTTATTGTAGGTGCTTTGGACTCTGATAAATATCCTTTGGTCAAGTGGCTAGATAGATCTACTGGAACATTTCTTGCTCCGGCTGCCCGTAATGACGTAATTCCTCTGGATAGCCTACAGTTTTTCATTGATTTTAAGAGGGAATGCCTATCGAAGGGCCTGCATCCCAGAGATTTACTGGGCTCGCCGATTACGGCTTTTGGGAAAATATGTACCACGTCGCGGCGCCTTAGACGCTTGCCAGGTGAAGAGTACGAGGTCGTACAGGGAATTAATTGTAGAAGGTGGCGCCTCCTGTGTGCCGAGGTAAAGGAATGCTGGTGGTGCGTTCATGCCAGGACTCACCTACACAGTGGGTCATCACTATGGGAAATTTTGTATCAACACAGTGTACGGCTCGAGAAGCATCGGAGAAGACCAAGGCCATTTGTGGGTGAAAACTCGGATTCCAGTGAGGAGGATCACCCAGCCTTTTGCGATGTGCCGGTCACGCAGACGGGCGCGGAATCTGAGGACTCTGGAGACGAGGGACCATCGACGCGCCATACTGCCTCTGGGGTTCAGCCAGTTGATGATGCCAATGCCGACTCTCCTGGCTCTGGAGACGAAGGACCCTCGACGCGTCATAGCGACTCGCAGCCCCCCCCGGCCGATGAACCAACGGTGCACACAGACAACGTTGAAGATGACCTCACACTGCTTGATAAAGAATCTGCATGTGCATTGATGTACCACGTGGGACAGGAGATGGACATGCTAATGAGGGCGATGTGCGATGAAGACCTCTTTGATCTGCTTGGCATCCCAGAGGATGTTATCGCAACATCACAGCCCGGAGGCGACACGGATGCAAGCGGCGTGGTAACAGAGGGCTCAATCGCCGCCTCGGCTGTCGGGGCGGGTGTAGAGGATGTGTACTTAGCTGGGGCACTCGAGGCCCAGAATGTAGCAGGGGAATATGTGTTGGAGATAAGTGACGAAGAAGTCGATGATGGTGCTGGACTGCCGCCGGCGTCCAGACGCCGGCCAGTTGTTGGCGAATTTTTATGGGATGATGGACCACGGAGACACGAGAGGCCTACCACGAGGCGCATTCGCCACAGGAAGCTTAGATCCGCATATTATAGAGTGGCACGGCCGCCAGTAATGATAACCGATAGGCTTGGTGTGGAAGTGTTTTATTTTGGCCGCCCTGCAATGTCTTTGGAAGTGGAACGAAAGGTGTTTATTCTATGTTCCCAGAACCCACTGGCAGACATTAGCCACTCTTGCTTGCATTCGCGCAAAGGGTTAAGAGTTTTGTTGCCCAAACCTGACGACAATAACACAGGGCCAGGAGACGTTAACCTGCTGGCGGCCGTGCTGCGCTCGTTTGCTTCGGGTCTTGTGATAGTTTCTCTCCGATCTGGCATTTATGTTAAGAATTTGTGCAAGTCTACCGTATTATATCATGGAAATAATCCTCCAAAGAAGTTTGGTGTGATCTGCGGACTGTCATCTAGGGCTGTTCTGGATGTTTTTAATGTGGCACAATATCGCATACAGGGACATGAGCACATTAAAAAAACAACTGTGTTCATCGGAGGTGACCCAACGTCGGCAGAACAGTTCGATATGGTCCCCCTCGTCATCAAGCTCAGATTGCGTTCAGTTACATGTGATGACTAA

SEQ ID NO: 3 - protein, 11 aa, synthetic construct
ACALMYHVGQE

SEQ ID NO: 4 - protein, 11 aa, synthetic construct
EMDMLMRAMCD

SEQ ID NO: 5 - protein, 11 aa, synthetic construct
DEDLFDLLGIP

SEQ ID NO: 6 - protein, 23 aa, synthetic construct
RRRQRRKKRGYGEQGVHYMLACA

SEQ ID NO: 7 - protein, 193 aa, synthetic construct
GSGDEGPSTRHSDSQPPPADEPTVHTDNVEDDLTLLDKESACALMYHVGQEMDMLMRAMCDEDLFDLLGIPEDVIATSQPGGDTDASGVVTEGSIAASAVGAGVEDVYLAGALEAQNVAGEYVLEISDEEVDDGAGLPPASRRRPVVGEFLWDDGPRRHERPTTRRIRHRKLRSAYYRVARPPVMITDRLGVE

SEQ ID NO: 8 - DNA, 32 bp, synthetic construct
  source 1..32: mol_type = other DNA
gcttgataaagaatctatggacatgctaatga

SEQ ID NO: 9 - DNA, 32 bp, synthetic construct
  source 1..32: mol_type = other DNA
tcattagcatgtccatagattctttatcaagc

SEQ ID NO: 10 - DNA, 32 bp, synthetic construct
  source 1..32: mol_type = other DNA
gcttgataaagaatctgaagacctctttgatc

SEQ ID NO: 11 - DNA, 29 bp, synthetic construct
  source 1..29: mol_type = other DNA
gatcaaaggtcttcagattctttatcaag

SEQ ID NO: 12 - DNA, 32 bp, synthetic construct
  source 1..32: mol_type = other DNA
gcttgataaagaatctgaggatgttatcgcaa

SEQ ID NO: 13 - DNA, 32 bp, synthetic construct
  source 1..32: mol_type = other DNA
ttgcgataacatcctcagattctttatcaagc

SEQ ID NO: 14 - DNA, 52 bp, synthetic construct
  source 1..52: mol_type = other DNA
gatccgccaccatggcatgtgcattgatgtaccacgtgggacaggagtaggc

SEQ ID NO: 15 - DNA, 52 bp, synthetic construct
  source 1..52: mol_type = other DNA
ggccgcctactcctgtcccacgtggtacatcaatgcacatgccatggtggcg

SEQ ID NO: 16 - DNA, 52 bp, synthetic construct
  source 1..52: mol_type = other DNA
gatccgccaccatggagatggacatgctaatgagggcgatgtgcgattaggc

SEQ ID NO: 17 - DNA, 51 bp, synthetic construct
  source 1..51: mol_type = other DNA
ggccgcctaatcgcacatcgccctcattagcatgtccatctccatggtgcg

SEQ ID NO: 18 - DNA, 52 bp, synthetic construct
  source 1..52: mol_type = other DNA
gatccgccaccatggatgaagacctctttgatctgcttggcatcccataggc

SEQ ID NO: 19 - DNA, 53 bp, synthetic construct
  source 1..53: mol_type = other DNA
ggccgcctatgggatgccaagcagatcaaagaggtcttcatccatgggtggcg